SEQUENCE LISTING (64 sequences)

SEQ ID NO: 1 - DNA, 654 bp, Homo sapiens
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 2 - DNA, 588 bp, Homo sapiens
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggttggccaaagacacatccaggaggggaacagaaggaggggacagaagcaagtctgcagatcagaggaaagaagaaagagcagaggagggagattggaagtagaaatgctgaatgcagaggcaaaaaaggaaaatga

SEQ ID NO: 3 - DNA, 333 bp, Homo sapiens
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 4 - DNA, 651 bp, Rattus norvegicus
atggctgcagactctcagactccctggctcctgaccttcagcctgctctgcctgctgtggcctcaagaggctggtgctttccctgccatgcccttgtccagtctgtttgccaatgctgtgctccgagcccagcacctgcaccagctggctgctgacacctacaaagagttcgagcgtgcctacattcccgagggacagcgctattccattcagaatgcccaggctgcgttctgcttctcagagaccatcccagcccccaccggcaaggaggaggcccagcagagaactgacatggaattgcttcgcttctcgctgctgctcatccagtcatggctggggcccgtgcagtttctcagcaggatctttaccaacagcctgatgtttggtacctcggaccgcgtctatgagaaactgaaggacctggaagagggcatccaggctctgatgcaggagctggaagacggcagcccccgtattgggcagatcctcaagcaaacctatgacaagtttgacgccaacatgcgcagcgatgacgctctgctcaaaaactatgggctgctctcctgcttcaagaaggacctgcacaaggcagagacctacctgcgggtcatgaagtgtcgccgctttgcggaaagcagctgtgctttctag

SEQ ID NO: 5 - DNA, 1551 bp, Homo sapiens
atggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtga

SEQ ID NO: 6 - DNA, 744 bp, Homo sapiens
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 7 - DNA, 804 bp, Homo sapiens
atgccgagatcgtgctgcagccgctcgggggccctgttgctggccttgctgcttcaggcctccatggaagtgcgtggctggtgcctggagagcagccagtgtcaggacctcaccacggatagcaacctgctggagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtag

SEQ ID NO: 8 - DNA, 1539 bp, Human alphaherpesvirus 1
atggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 9 - DNA, 183 bp, Mesobuthus eupeus
atgaaattcctctatggaatcgtttttattgctctttttttaacagtaatgatcgcaactcatactgaagctatgtgtatgccttgctttacaacccgtcctgatatggcacagcagtgtagagactgttgcggaggcaatggaaaatgctttggctatcaatgtttgtgtaatcgtggttga

SEQ ID NO: 10 - protein, 60 aa, Mesobuthus eupeus
MKFLYGIVFIALFLTVMIATHTEAMCMPCFTTRPDMAQQCRDCCGGNGKCFGYQCLCNRG

SEQ ID NO: 11 - DNA, 954 bp, Homo sapiens
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 12 - DNA, 1137 bp, Homo sapiens
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagggagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcggtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagagcctgtctgcctcctgctgccatcaacctgctgtctatagctcccatggcacccaggctgttcatgccaaagggtgcctgcaggcccacggcacagaaagggagtcacaagacactgttgcagagaatgatgatggcgggttcagtgaggaatgggaagcccagagggacagtcatctag

SEQ ID NO: 13 - DNA, 1344 bp, Homo sapiens
atgctcacattcatggcctctgacagcgaggaagaagtgtgtgatgagcggacgtccctaatgtcggccgagagccccacgccgcgctcctgccaggagggcaggcagggcccagaggatggagagaatactgcccagtggagaagccaggagaacgaggaggacggtgaggaggaccctgaccgctatgtctgtagtggggttcccgggcggccgccaggcctggaggaagagctgaccctcaaatacggagcgaagcatgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccatggacaacctggtgcggccgttcatggacaccctggcctcccatcagctctacatc

SEQ ID NO: 14 - DNA, 1323 bp, Homo sapiens
atggaatatcagatattgaaaatgtctctctgcctgttcatccttctgtttctcacacctggtattttatgcatttgtcctctccaatgtatatgcacagagaggcacaggcatgtggactgttcaggcagaaacttgtctacattaccatctggactgcaagagaatattatacatttaaacctgtcttataaccactttactgatctgcataaccagttaacccaatataccaatctgaggaccctggacatttcaaacaacaggcttgaaagcctgcctgctcacttacctcggtctctgtggaacatgtctgctgctaacaacaacattaaacttcttgacaaatctgatactgcttatcagtggaatcttaaatatctggatgtttctaagaacatgctggaaaaggttgtcctcattaaaaatacactaagaagtctcgaggttctcaacctcagtagtaacaaactttggacagttccaaccaacatgccctccaaactacatatcgtggacctgtctaataattctttgacacaaattcttccaggtacattaataaacctgacaaatctcacacatctttacctgcacaacaataagttcacattcattccagaccaatcttttgaccaactctttcagttgcaagagataaccctttacaataacaggtggtcatgtgaccacaaacaaaacattacttacttactgaagtggatgatggaaacaaaagcccatgtgatagggactccatgttctacccaaatatcatctttaaaggaacataacatgtatcccacaccttctggatttacctcaagcttattcactgtaagtgggatgcagacagtggacaccattaactctctgagtgtggtaactcaacccaaagtgaccaaaatacccaaacaatatcgaacaaaggaaacaacgtttggtgccactctaagcaaagacaccacctttactagcactgataaggcttttgtgccctatccagaagatacatccacagagactatcaattcacatgaagcagcagctgcaactctaactattcatctccaagatggaatggtcacaaacacaagcctcactagctcaacaaaatcatccccaacacccatgaccctaagtatcactagtggcatgccaaataatttctctgaaatgcctcaacaaagcacaacccttaacttatggagggaagagacaaccacaaatgtaaagactccattaccttctgtggcaaatgcttggaaagtaaatgcttcatttctcttattgctcaatgttgtggtcatgctggctgtctga

SEQ ID NO: 15 - DNA, 180 bp, Homo sapiens
gtcagggtaagtttctgccaaggtttccttttgctaagcctaaaattcagagtaaccaatcttaagtttgctgttgagatatcttgcaaatacaaatgctttcttgggtatatcaagaggcattctttatcacctccctagagatcttcaagtggtcagatgctcagcagttctgtaaag

SEQ ID NO: 16 - DNA, 1327 bp, Homo sapiens
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtactggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggactggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgt

SEQ ID NO: 17 - DNA, 1758 bp, Homo sapiens
atggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataa

SEQ ID NO: 18 - DNA, 537 bp, Homo sapiens
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 19 - DNA, 3969 bp, Homo sapiens
atggccacatcgaatccggctttcgaccccaaaaacttaatgcagagtgaaatctatcattttgcccagaataacccattggcagacttttcaagcgataaaaactccattctgacgttatctgacaagcgttccattatgggaaatcaatccttactttggaaatggaaaggcggctccagttttacgttacataaaaaattaatcgtgccgaccgataaggaagcatctaaagcatggggacgcagtagtacgccggtgttctccttctggctgtacaacgagaaacctattgatgggtacttgaccatcgacttcggcgaaaaattgatttcaactagcgaagcacaagctggctttaaggttaaattggacttcaccgggtggcgtacggtcggagtcagccttaacaatgacttagagaatcgcgaaatgaccttgaatgctacgaatacgtccagcgatggaacccaggatagcatcggtcgtagtctgggagcaaaggtggacagtatccgcttcaaagctccgagcaacgtttcacagggggaaatttatatcgaccgtattatgttcagtgtcgacgacgcccgttatcaatggagtgactatcaagtaaagacccgtctgtcagaacctgagatccagtttcataatgtgaagccgcagttgcccgtaacgcccgaaaatttggcagctattgatcttatccgccagcgcctgatcaatgagtttgtcggcggggagaaagaaaccaacctggcgctggaagaaaatatctcaaaactgaaaagtgactttgatgcacttaatatccacacgctggcgaacgggggaacccaaggacgtcacttaattactgacaagcaaattatcatctatcaacccgagaatcttaactcacaagataaacagttatttgacaattatgtcatcttaggtaactacacgaccttgatgtttaacatctctcgcgcctatgttttggaaaaagaccccactcaaaaggcacaattaaagcagatgtaccttctgatgaccaaacatcttttggatcagggattcgtgaaaggtagtgcgttagtaacaacacaccattgggggtacagttcgcgctggtggtatatcagtactttgttaatgtctgatgcattgaaggaggcgaatcttcagacgcaggtttacgattccctgctgtggtattcgcgtgagttcaaatcttcgttcgatatgaaggtcagcgctgactcttcggatttggattactttaataccctttcgcgccagcacttggccttgttactgctggagccagacgaccagaaacgcatcaaccttgtcaacacgtttagtcactatatcacaggagcgcttactcaagtaccgccggggggaaaagatggtcttcgtccagatggtacggcctggcgccatgagggtaactatccgggctattcctttccggcatttaagaatgcgtcccaactgatttaccttctgcgtgataccccattttcagtgggagaatctggatggaacaacttgaagaaagcgatggtctctgcttggatttactctaacccagaagttggattaccattagcaggccgtcacccattcaatagtccctcccttaaaagtgtcgcccaggggtattattggttagctatgtctgcaaagtcgagtcctgacaaaaccctggcgtctatctacttggccatctccgataaaactcaaaacgaatcaaccgccatttttggcgaaaccatcacgccggcttctttgccgcaggggttctacgcctttaatggaggcgccttcggcattcatcgttggcaggataagatggttacattgaaagcgtacaataccaacgtatggagttccgagatctataacaaggataatcgctatggccgttatcagtctcacggggtggctcaaattgtctctaacggtagtcaattaagtcaaggttatcaacaggagggatgggactggaaccgcatgcctggggcaacaactatccatctgccgctgaaggatttagatagccctaagccacatacactgatgcagcgtggtgaacgtggcttctcgggtactagttcactggaggggcaatacgggatgatggcatttgacttaatctatccagctaatttagagcgcttcgaccccaattttactgctaagaagagcgtcctggcagcggataaccacctgatctttattggaagcaatattaatagctcggacaagaacaagaatgttgagacgacactttttcagcatgccatcactccaactttgaatacgctgtggatcaatggacaaaagatcgaaaatatgccataccagacaacacttcagcagggtgactggttgattgactcgaatgggaacgggtacttaattacacaagccgaaaaagtgaatgtctcgcgccagcaccaagtttcggcggagaataaaaaccgtcaacctactgagggcaacttcagctctgcctggattgatcacagtactcgcccaaaagacgcatcatatgagtacatggtattcttggatgctacgcctgagaaaatgggagagatggctcaaaagttccgtgagaacaacggattgtaccaagttctgcgcaaggataaagatgtgcatatcattttagataaactttccaatgtaacggggtatgccttttatcaacccgccagtattgaggataaatggatcaagaaagtgaataagcccgccatcgtaatgactcatcgccagaaagacactcttattgtctcggcggtaacacccgatcttaatatgactcgccagaaagcagctactcccgtaacaatcaacgttacgatcaatgggaagtggcaaagtgctgataagaacagtgaggttaaataccaggtgtccggcgataatacggagttgacgtttacatcttacttcggcatcccacaagaaattaagttgtcccctttgccgggatccggcggcggcagcggcggcagcggcggcagcggcggcatggcgcgcatgaaacagctggaagataaagtggaagaactgctgagcaaaaactatcatctggaaaaccgcgtggcgcgcctggaaaaactggtgggcgaacgcggcggcggcagcggcggcagcggcggcggcggcagcggcggcagcggcgaattcgcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgcccccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataacatgatactcgagcaccaccaccaccaccactga

SEQ ID NO: 20 - DNA, 1224 bp, Homo sapiens
atggataacgtcctcccggtggactcagacctctccccaaacatctccactaacacctcggaacccaatcagttcgtgcaaccagcctggcaaattgtcctttgggcagctgcctacacggtcattgtggtgacctctgtggtgggcaacgtggtagtgatgtggatcatcttagcccacaaaagaatgaggacagtgacgaactattttctggtgaacctggccttcgcggaggcctccatggctgcattcaatacagtggtgaacttcacctatgctgtccacaacgaatggtactacggcctgttctactgcaagttccacaacttctttcccatcgccgctgtcttcgccagtatctactccatgacggctgtggcctttgataggtacatggccatcatacatcccctccagccccggctgtcagccacagccaccaaagtggtcatctgtgtcatctgggtcctggctctcctgctggccttcccccagggctactactcaaccacagagaccatgcccagcagagtcgtgtgcatgatcgaatggccagagcatccgaacaagatttatgagaaagtgtaccacatctgtgtgactgtgctgatctacttcctccccctgctggtgattggctatgcatacaccgtagtgggaatcacactatgggccagtgagatccccggggactcctctgaccgctaccacgagcaagtctctgccaagcgcaaggtggtcaaaatgatgattgtcgtggtgtgcaccttcgccatctgctggctgcccttccacatcttcttcctcctgccctacatcaacccagatctctacctgaagaagtttatccagcaggtctacctggccatcatgtggctggccatgagctccaccatgtacaaccccatcatctactgctgcctcaatgacaggttccgtctgggcttcaagcatgccttccggtgctgccccttcatcagcgccggcgactatgaggggctggaaatgaaatccacccggtatctccagacccagggcagtgtgtacaaagtcagccgcctggagaccaccatctccacagtggtgggggcccacgaggaggagccagaggacggccccaaggccacaccctcgtccctggacctgacctccaactgctcttcacgaagtgactccaagaccatgacagagagcttcagcttctcctccaatgtgctctcctag

SEQ ID NO: 21 - DNA, 762 bp, Homo sapiens
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 22 - DNA, 1083 bp, Homo sapiens
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 23 - DNA, 952 bp, Homo sapiens
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgt

SEQ ID NO: 24 - DNA, 1374 bp, Homo sapiens
atggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactga

SEQ ID NO: 25 - DNA, 918 bp, Homo sapiens
atgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtga

SEQ ID NO: 26 - DNA, 630 bp, Homo sapiens
atgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 27 - DNA, 1863 bp, Homo sapiens
atggcagaggcccgcacatccctgtctgcccactgtcggggcccgctggccactggcctgcacccagacctggacctcccgggccgaagcctcgccacccctgcgccttcctgctaccttctgggcagcgaacccagctctggcctgggcctccagcccgagacccacctccccgagggcagcctgaagcggtgctgcgtcttgggcctaccccccacctccccagcctcctcctcaccctgtgcctcctccgacgtcacctccatcatccgctcctcccagacgtctctggtcacctgtgtaaatggactccggagcccccctctgacgggagatctggggggcccttccaagcgggcccggcctggccctgcatcgacggacagccatgagggcagcttgcaacttgaagcctgccggaaggcgagcttcctgaagcaggaacccgcggatgagttttcagagctctttgggcctcaccagcagggcctgccgcccccctatcccctgtctcagttgccgcctggcccaagccttggaggcctggggctgggcctggcaggcagggtggtggccgggcggcaggcgtgccgctgggtggactgctgtgcagcctatgagcagcaggaggagctggtgcggcacatcgagaagagccacatcgaccagcgcaagggcgaggacttcacctgcttctgggctggctgcgtgcgccgctacaagcccttcaacgcccgctacaagctgctcatccacatgcgagtgcactcgggcgagaagcccaacaagtgcatgtttgaaggctgcagcaaggccttctcacggctggagaacctcaagatccacctgaggagccacacgggcgagaagccgtacctgtgccagcacccgggttgccagaaggccttcagcaactccagcgaccgcgccaagcaccagcgcacccacctagacacgaagccgtacgcctgtcagatccctggctgctccaagcgctacacagaccccagctccctccgcaagcacgtcaaggcccattcagccaaagagcagcaggtgcgtaagaagctgcatgcgggccctgacaccgaggccgacgtcctgaccgagtgtctggtcctgcagcagctccacacgtccacacagctggctgccagcgacggcaagggtggctgtggcctgggccaggagctgctcccaggtgtgtatcctggctccatcaccccccataacggacttgcatcgggcctcctgcccccagcgcacgacgtaccttccaggcaccacccgctggatgccaccaccagttcccaccaccatctgtcccctctgcccatggctgagagcacccgggatgggttggggcccggcctcctctcaccaatagtcagccccctgaaggggctggggccaccgccgctgcccccatcctctcagagccattctccggggggccagcccttccccacactccccagcaagccgtcctacccacccttccagagccctccacccccgcctctgcccagcccacaaggttaccagggcagtttccactccatccagagttgcttcccctatggcgactgctaccggatggctgaaccagcagccggtggggacggactggtcggggagacccacggtttcaaccccctgcggcccaatggctaccacagcctcagcacgcccttgcctgccacaggctatgaggccctggctgaggcctcatgccccacagcgctgccacagcagccatctgaagatgtggtgtccagcggccccgaggactgtggcttcttccccaatggagcctttgaccactgcctgggccacatcccctccatctacacagacacctga

SEQ ID NO: 28 - DNA, 711 bp, Homo sapiens
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctga

SEQ ID NO: 29 - DNA, 1149 bp, Homo sapiens
atgctggacggcctaaagatggaggagaacttccaaagcgcgatcgacacctcggcctccttctcctcgctgctgggcagagcggtgagccccaagtctgtctgcgagggctgtcagcgggtcatcttggacaggtttctgctgcggctcaacgacagcttctggcatgagcagtgcgtgcagtgcgcctcctgcaaagagcccctggagaccacctgcttctaccgggacaagaagctgtactgcaagtatgactacgagaagctgtttgctgttaaatgtgggggctgcttcgaggccatcgctcccaatgagtttgttatgcgggcccagaagagtgtataccacctgagctgcttctgctgctgtgtctgcgagcgacagcttcagaagggtgatgagtttgtcctgaaggaggggcagctgctctgcaaaggggactatgagaaggagcgggagctgctcagcctggtgagcccagcagcctcagactcaggtaaaagtgatgatgaagaaagtctctgcaagtcagcccatggggcagggaaaggaactgctgaggaaggcaaggaccataagcgccccaaacgtccgagaaccatcttgacaactcaacagaggcgagcattcaaggcctcatttgaagtatcctccaagccctgcaggaaggtgagagagactctggctgcagagacagggctgagtgtccgtgtcgtccaggtgtggttccaaaaccagagagcgaagatgaagaagctggccaggcgacagcagcagcagcagcaagatcagcagaacacccagaggctgagctctgctcagacaaacggtggtgggagtgctgggatggaaggaatcatgaacccctacacggctctgcccaccccacagcagctcctggccatcgagcagagtgtctacagctcagatcccttccgacagggtctcaccccaccccagatgcctggagaccacatgcacccttatggtgccgagccccttttccatgacctggatagcgacgacacctccctcagtaacctgggtgattgtttcctagcaacctcagaagctgggcctctgcagtccagagtgggaaaccccattgaccatctgtactccatgcagaattcttacttcacatcttga

SEQ ID NO: 30 - DNA, 3561 bp, Homo sapiens
atggaggtggacaccgaggagaagcggcatcgcacgcggtccaaaggggttcgagttcccgtggaaccagccatacaagagctgttcagctgtcccacccctggctgtgacggcagtggtcatgtcagtggcaaatatgcaagacacagaagtgtatatggttgtcccttggcgaaaaaaagaaaaacacaagataaacagccccaggaacctgctcctaaacgaaagccatttgccgtgaaagcagacagctcctcagtggatgagtgtgacgacagtgatgggactgaggacatggatgagaaggaggaggatgagggggaggagtactccgaggacaatgatgagccaggggatgaggacgaggaggacgaggagggggaccgggaggaggaggaggagatcgaggaggaggatgaggacgatgacgaggatggagaagatgtggaggatgaagaagaggaagaggaggaggaggaggaggaggaagaggaagaagaaaacgaagaccatcaaatgaattgtcacaatactcgaataatgcaagacacagaaaaggatgataacaataatgacgaatatgacaattacgatgaactggtggccaagtcattgttaaacctcggcaaaatcgctgaggatgcagcctaccgggccaggactgagtcagaaatgaacagcaatacctccaatagtctggaagacgatagtgacaaaaacgaaaacctgggtcggaaaagtgagttgagtttagacttagacagtgatgttgttagagaaacagtggactcccttaaactattagcccaaggacacggtgttgtgctctcagaaaacatgaatgacagaaattatgcagacagcatgtcgcagcaagacagtagaaatatgaattacgtcatgttggggaagcccatgaacaacggactcatggaaaagatggtggaggagagcgatgaggaggtgtgtctgagcagtctggagtgtttgaggaatcagtgcttcgacctggccaggaagctcagtgagaccaacccgcaggagaggaatccgcagcagaacatgaacatccgtcagcatgtccggccagaagaggacttccccggaaggacgccggacagaaactactcggacatgctgaacctcatgcggctggaggagcagttgagcccccggtcgagagtgtttgccagctgtgcgaaggaggatgggtgtcatgagcgggacgacgataccacctctgtgaactcggacaggtctgaagaggtgttcgacatgaccaaggggaacctgaccctgctggagaaagccatcgctttggaaacggaaagagcaaaggccatgagggagaagatggccatggaagctgggaggagggacaatatgaggtcatatgaggaccagtctccgagacaacttcccggggaggacagaaagcctaaatccagtgacagccatgtcaaaaagccatactatggtaaagatccctcaagaacagaaaagaaagagagcaagtgtccaacccccgggtgtgatggaaccggccacgtaactgggctgtacccacatcaccgcagcctgtccggatgcccgcacaaagatagggtccctccagaaatccttgccatgcatgaaagtgtcctcaagtgccccactccgggctgcacggggcgcgggcatgtcaacagcaacaggaactcccaccgaagcctctccggatgcccgatcgctgcagcagagaaactggccaaggcacaggaaaagcaccagagctgcgacgtgtccaagtccagccaggcctcggaccgcgtgctcaggccaatgtgctttgtgaagcagctggagattcctcagtatggctacagaaacaatgtccccacaactacgccgcgttccaacctggccaaggagctcgagaaatattccaagacctcgtttgaatacaacagttacgacaaccatacttatggcaagcgagccatagctcccaaggtgcaaaccagggatatatcccccaaaggatatgatgatgcgaagcggtactgcaaggaccccagccccagcagcagcagcaccagcagctacgcgcccagcagcagcagcaacctgagctgcggcgggggcagcagcgccagcagcacgtgcagcaagagcagcttcgactacacgcacgacatggaggcggcccacatggcggccaccgccatcctcaacctgtccacgcgctgccgcgagatgccgcagaacctgagcaccaagccgcaggacctgtgcgccacgcggaaccctgacatggaggtggatgagaacgggaccctggacctcagcatgaacaagcagaggccgcgggacagctgctgccccatcctgacccctctggagcccatgtccccccagcagcaggcagtgatgaacaaccggtgtttccagctgggcgagggcgactgctgggacttgcccgtagactacaccaaaatgaaaccccggaggatagacgaggacgagtccaaagacattactccagaagacttggacccattccaggaggctctagaagaaagacggtatcccggggaggtgaccatcccaagtcccaaacccaagtaccctcagtgcaaggagagcaaaaaggacttaataactctgtctggctgccccctggcggacaaaagcattcgaagtatgctggccaccagctcccaagaactcaagtgccccacgcctggctgtgatggttctggacatatcaccggcaattatgcttctcatcggagcctttcaggttgcccaagagcaaagaaaagtggtatcaggatagcacagagcaaagaagataaagaagatcaagaacccatcaggtgtccggtccccgggtgcgacggccagggccacatcactgggaagtacgcgtcccatcgcagcgcctccgggtgccccttggcggccaagaggcagaaagacgggtacctgaatggctcccagttctcctggaagtcggtcaagacggaaggcatgtcctgccccacgccaggatgcgacggctcaggccacgtcagcggcagcttcctcacacaccgcagcttgtcaggatgcccgagagccacgtcagcgatgaagaaggcaaagctttctggagagcagatgctgaccatcaaacagcgggccagcaacggtatagaaaatgatgaagaaatcaaacagttagatgaagaaatcaaggagctaaatgaatccaattcccagatggaagccgatatgattaaactcagaactcagattaccacgatggagagcaacctgaagaccatcgaagaggagaacaaagtgattgagcagcagaacgagtctctcctccacgagctggcgaacctgagccagtctctgatccacagcctggctaacatccagctgccgcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctga

SEQ ID NO: 31 - DNA, 1332 bp, Homo sapiens
atggcgaccgcagcgtctaaccactacagcctgctcacctccagcgcctccatcgtgcacgccgagccgcccggcggcatgcagcagggcgcggggggctaccgcgaagcgcagagcctggtgcagggcgactacggcgctctgcagagcaacggacacccgctcagccacgctcaccagtggatcaccgcgctgtcccacggcggcggcggcgggggcggtggcggcggcggggggggcgggggcggcggcgggggcggcggcgacggctccccgtggtccaccagccccctgggccagccggacatcaagccctcggtggtggtgcagcagggcggccgcggagacgagctgcacgggccaggcgccctgcagcagcagcatcagcagcagcaacagcaacagcagcagcaacagcagcaacagcagcagcagcagcagcaacagcggccgccgcatctggtgcaccacgccgctaaccaccacccgggacccggggcatggcggagcgcggcggctgcagcgcacctcccaccctccatgggagcgtccaacggcggcttgctctactcgcagcccagcttcacggtgaacggcatgctgggcgccggcgggcagccggccggtctgcaccaccacggcctgcgggacgcgcacgacgagccacaccatgccgaccaccacccgcacccgcactcgcacccacaccagcagccgccgcccccgccgcccccgcagggtccgcctggccacccaggcgcgcaccacgacccgcactcggacgaggacacgccgacctcggacgacctggagcagttcgccaagcagttcaagcagcggcggatcaaactgggatttacccaagcggacgtggggctggctctgggcaccctgtatggcaacgtgttctcgcagaccaccatctgcaggtttgaggccctgcagctgagcttcaagaacatgtgcaagctgaagcctttgttgaacaagtggttggaggaggcggactcgtcctcgggcagccccacgagcatagacaagatcgcagcgcaagggcgcaagcggaaaaagcggacctccatcgaggtgagcgtcaagggggctctggagagccatttcctcaaatgccccaagccctcggcccaggagatcacctccctcgcggacagcttacagctggagaaggaggtggtgagagtttggttttgtaacaggagacagaaagagaaaaggatgacccctcccggagggactctgccgggcgccgaggatgtgtacggggggagtagggacactccaccacaccacggggtgcagacgcccgtccagtga

SEQ ID NO: 32 - DNA, 1071 bp, Homo sapiens
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 33 - DNA, 1269 bp, Homo sapiens
atgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaa

SEQ ID NO: 34 - DNA, 1041 bp, Homo sapiens
atgcacctgccgggctgcgcgccagccatggccgacgggagcttctcgcttgccggccacctgctccgcagcccgggcgggagcacctcgcgacttcacagcatcgaggccatcctggggtttaccaaggacgacgggatcctcggcaccttcccggcggagcggggcgcccggggcgcgaaggagcgggataggaggctgggcgcgcggcccgcctgccccaaggcgcccgaggaaggctccgagccctccccgccgccagccccggcgcccgcccccgagtacgaagcccctcgaccctactgccccaaggagcccggggaggcacggccgagcccagggctgcccgtcgggccagccaccggcgaagcgaaactgtcagaggaggaacagcccaagaaaaagcatcggcggaaccgcacgactttcaccacgtaccagctgcatgagctggagcgcgcgttcgagaagtcccactacccggacgtgtacagccgcgaggagctggccggcaaggtcaacctaccagaggtccgggtccaggtgtggttccagaaccgacgggctaagtggcggcggcaggagaagctggaagtgtcctccatgaagctgcaggactcgcccctcctctccttcagccgctccccgccctccgcgacgctgtcgcccctcggggcgggcccgggcagcggtggcgggccggctgggggcgcgctgccgctggagtcctggctcgggccgccgctgccgggcgggggcgccacggcgctgcagagcctgccgggcttcgggccgccggcgcagagcctgcctgccagctacacgccaccgccgccgcctccgcccttcctgaactccccgccgttgggccccggcctgcaacctctcgcgccgccgccgccctcctacccgtgcgggcccggcttcggggacaagttcccgctggacgaggcggacccgcgcaacagcagcatcgcggcgctgcgtctgaaagccaaggagcacatccaggccatcgggaagccgtggcaggccctctag

SEQ ID NO: 35 - DNA, 900 bp, Homo sapiens
atgatggcgtatatgaacccggggccccactattctgtcaacgccttggccctaagtggccccagtgtggatctgatgcaccaggctgtgccctacccaagcgcccccaggaagcagcggcgggagcgcaccaccttcacccggagccaactggaggagctggaggcactgtttgccaagacccagtacccagacgtctatgcccgtgaggaggtggctctgaagatcaatctgcctgagtccagggttcaggtttggttcaagaaccggagggctaaatgcaggcagcagcgacagcagcagaaacagcagcagcagcccccagggggccaggccaaggcccggcctgccaagaggaaggcgggcacgtccccaagaccctccacagatgtgtgtccagaccctctgggcatctcagattcctacagtccccctctgcccggcccctcaggctccccaaccacggcagtggccactgtgtccatctggagcccagcctcagagtcccctttgcctgaggcgcagcgggctgggctggtggcctcagggccgtctctgacctccgccccctatgccatgacctacgccccggcctccgctttctgctcttccccctccgcctatgggtctccgagctcctatttcagcggcctagacccctacctttctcccatggtgccccagctagggggcccggctcttagccccctctctggcccctccgtgggaccttccctggcccagtcccccacctccctatcaggccagagctatggcgcctacagccccgtggatagcttggaattcaaggaccccacgggcacctggaaattcacctacaatcccatggaccctctggactacaaggatcagagtgcctggaagtttcagatcttgtag

SEQ ID NO: 36 - DNA, 1563 bp, Homo sapiens
atgcagtccgaatcggggatcgtgccggatttcgaagtcggggaggagtttcatgaagagcccaaaacctattacgaactcaaaagtcaaccgctgaagagcagcagttccgccgagcatcctggggcctccaagcctccgataagctcctccagtatgacatcacgcatcttgctacgccagcaactcatgcgtgagcagatgcaggagcaggagcgcagggagcagcagcagaagctgcaggcggcccagttcatgcaacagagagtgcccgtgagtcagacaccagccataaacgtcagtgtgcccaccacccttccctctgccacgcaggtgccgatggaagtccttaaggtgcagacccacctcgaaaaccccaccaagtaccacatacagcaagcccaacggcagcaggtaaagcagtacctttctaccactttagcaaataaacatgccaaccaagtcctgagcttgccatgtccaaaccagcctggcgatcatgtcatgccaccggtgccggggagcagcgcacccaacagccccatggctatgcttacgcttaactccaactgtgaaaaagagggattttataagtttgaagagcaaaacagggcagagagcgagtgcccaggcatgaacacacattcacgagcgtcctgtatgcagatggatgatgtaatcgatgacatcattagcctagaatcaagttataatgaggaaatcttgggcttgatggatcctgctttgcaaatggcaaatacgttgcctgtctcgggaaacttgattgatctttatggaaaccaaggtctgcccccaccaggcctcaccatcagcaactcctgtccagccaaccttcccaacataaaaagggagctcacagagtctgaagcaagagcactggccaaagagaggcagaaaaaggacaatcacaacctgattgaacgaagaagaagatttaacataaatgaccgcattaaagaactaggtactttgattcccaagtcaaatgatccagacatgcgctggaacaagggaaccatcttaaaagcatccgtggactatatccgaaagttgcaacgagaacagcaacgcgcaaaagaacttgaaaaccgacagaagaaactggagcacgccaaccggcatttgttgctcagaatacaggaacttgaaatgcaggctcgagctcatggactttcccttattccatccacgggtctctgctctccagatttggtgaatcggatcatcaagcaagaacccgttcttgagaactgcagccaagacctccttcagcatcatgcagacctaacctgtacaacaactctcgatctcacggatggcaccatcaccttcaacaacaacctcggaactgggactgaggccaaccaagcctatagtgtccccacaaaaatgggatccaaactggaagacatcctgatggacgacaccctttctcccgtcggtgtcactgatccactcctttcctcagtgtcccccggagcttccaaaacaagcagccggaggagcagtatgagcatggaagagacggagcacacttgttag

SEQ ID NO: 37 - DNA, 894 bp, Homo sapiens
atgatgtcttatcttaagcaaccgccttacgcagtcaatgggctgagtctgaccacttcgggtatggacttgctgcacccctccgtgggctacccggggccctgggcttcttgtcccgcagccaccccccggaaacagcgccgggagaggacgacgttcactcgggcgcagctagatgtgctggaagcactgtttgccaagacccggtacccagacatcttcatgcgagaggaggtggcactgaaaatcaacttgcccgagtcgagggtgcaggtatggtttaagaatcgaagagctaagtgccgccaacaacagcaacaacagcagaatggaggtcaaaacaaagtgagacctgccaaaaagaagacatctccagctcgggaagtgagttcagagagtggaacaagtggccaattcactcccccctctagcacctcagtcccgaccattgccagcagcagtgctcctgtgtctatctggagcccagcttccatctccccactgtcagatcccttgtccacctcctcttcctgcatgcagaggtcctatcccatgacctatactcaggcttcaggttatagtcaaggatatgctggctcaacttcctactttgggggcatggactgtggatcatatttgacccctatgcatcaccagcttcccggaccaggggccacactcagtcccatgggtaccaatgcagtcaccagccatctcaatcagtccccagcttctctttccacccagggatatggagcttcaagcttgggttttaactcaaccactgattgcttggattataaggaccaaactgcctcctggaagcttaacttcaatgctgactgcttggattataaagatcagacatcctcgtggaaattccaggttttgtga

SEQ ID NO: 38 - DNA, 714 bp, Homo sapiens
atggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctga

SEQ ID NO: 39 - DNA, 1440 bp, Homo sapiens
atgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 40 - DNA, 819 bp, Homo sapiens
atgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggagccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctag

SEQ ID NO: 41 - DNA, 1326 bp, Homo sapiens
atggtgcagcaggcggagagcttggaagcggagagcaacctgccccgggaggcgctggacacggaggagggcgaattcatggcttgcagcccggtggccctggacgagagcgacccagactggtgcaagacggcgtcgggccacatcaagcggccgatgaacgcgttcatggtatggtccaagatcgaacgcaggaagatcatggagcagtctccggacatgcacaacgccgagatctccaagaggctgggcaagcgctggaaaatgctgaaggacagcgagaagatcccgttcatccgggaggcggagcggctgcggctcaagcacatggccgactaccccgactacaagtaccggccccggaaaaagcccaaaatggacccctcggccaagcccagcgccagccagagcccagagaagagcgcggccggcggcggcggcgggagcgcgggcggaggcgcgggcggtgccaagacctccaagggctccagcaagaaatgcggcaagctcaaggcccccgcggccgcgggcgccaaggcgggcgcgggcaaggcggcccagtccggggactacgggggcgcgggcgacgactacgtgctgggcagcctgcgcgtgagcggctcgggcggcggcggcgcgggcaagacggtcaagtgcgtgtttctggatgaggacgacgacgacgacgacgacgacgacgagctgcagctgcagatcaaacaggagccggacgaggaggacgaggaaccaccgcaccagcagctcctgcagccgccggggcagcagccgtcgcagctgctgagacgctacaacgtcgccaaagtgcccgccagccctacgctgagcagctcggcggagtcccccgagggagcgagcctctacgacgaggtgcgggccggcgcgacctcgggcgccgggggcggcagccgcctctactacagcttcaagaacatcaccaagcagcacccgccgccgctcgcgcagcccgcgctgtcgcccgcgtcctcgcgctcggtgtccacctcctcgtccagcagcagcggcagcagcagcggcagcagcggcgaggacgccgacgacctgatgttcgacctgagcttgaatttctctcaaagcgcgcacagcgccagcgagcagcagctggggggcggcgcggcggccgggaacctgtccctgtcgctggtggataaggatttggattcgttcagcgagggcagcctgggctcccacttcgagttccccgactactgcacgccggagctgagcgagatgatcgcgggggactggctggaggcgaacttctccgacctggtgttcacatattga

SEQ ID NO: 42 - DNA, 1050 bp, Homo sapiens
atgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatga

SEQ ID NO: 43 - DNA, 1194 bp, Homo sapiens
atgctgctggaaacggggctcgagcgcgaccgagcgaggcccggggccgccgccgtctgcaccttgggcgggactcgggagatcccgctgtgcgctggctgtgaccagcacatcctggaccgcttcatcctcaaggctctggaccgccactggcacagcaagtgtctcaagtgcagcgactgccacacgccactggccgagcgctgcttcagccgaggggagagcgtttactgcaaggacgactttttcaagcgcttcgggaccaagtgcgccgcgtgccagctgggcatcccgcccacgcaggtggtgcgccgcgcccaggacttcgtgtaccacctgcactgctttgcctgcgtcgtgtgcaagcggcagctggccacgggcgacgagttctacctcatggaggacagccggctcgtgtgcaaggcggactacgaaaccgccaagcagcgagaggccgaggccacggccaagcggccgcgcacgaccatcaccgccaagcagctggagacgctgaagagcgcttacaacacctcgcccaagccggcgcgccacgtgcgcgagcagctctcgtccgagacgggcctggacatgcgcgtggtgcaggtttggttccagaaccgccgggccaaggagaagaggctgaagaaggacgccggccggcagcgctgggggcagtatttccgcaacatgaagcgctcccgcggcggctccaagtcggacaaggacagcgttcaggaggggcaggacagcgacgctgaggtctccttccccgatgagccttccttggcggaaatgggcccggccaatggcctctacgggagcttgggggaacccacccaggccttgggccggccctcgggagccctgggcaacttctccctggagcatggaggcctggcaggcccagagcagtaccgagagctgcgtcccggcagcccctacggtgtccccccatcccccgccgccccgcagagcctccctggcccccagcccctcctctccagcctggtgtacccagacaccagcttgggccttgtgccctcgggagcccccggcgggcccccacccatgagggtgctggcagggaacggacccagttctgacctatccacggggagcagcgggggttaccccgacttccctgccagccccgcctcctggctggatgaggtagaccacgctcagttctga

SEQ ID NO: 44 - DNA, 1350 bp, Homo sapiens
gaagtgcagctgctggaatctggcggcggactggtgcagcctggcggctctctgagactgtcttgtgccgcctccggctacaccttcaccacctactggatcacctgggtccgacaggctcccggcaagggactggaatgggtcggagacatctaccccggctcctccatctgcaactacgccgacaagttcaagggccggttcaccatctccgtggacaactccaagtccaccgcctacctccagatgaactccctgagagccgaggacaccgccgtgtactactgcgccagagaggacggctacgacgcttggtttgcttactggggccagggcaccctggtcaccgtgtcatctgcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 45 - DNA, 396 bp, Homo sapiens
atgaagttgcctgttaggctgttggtgctgatgttctggattcctggttccagcagtgatgttatgatgacccaaactccactctccctgcctgtcagtcttggagatcaagcctccatctcttgcagatctagtcagagtcttgtacatagtaatggaaacacctatttagaatggtatatgcagaaaccaggccagtctccaatgctcctgatctacaaagtttccaaccgattttctggggtcccagacaggttcagtggcagtggatcagggacagatttcacactcaagatcagcagcgtggaggctgaggatctgggagttttttactgctttcaaggttcacgtgttccgctcacgttcggtgctgggaccaagctggagctgaaacgg

SEQ ID NO: 46 - protein, 11 aa, Homo sapiens
RQIKIWFQNRR

SEQ ID NO: 47 - protein, 11 aa, Homo sapiens
YGRKKRRQRRR

SEQ ID NO: 48 - protein, 21 aa, Homo sapiens
LLNFDLLKILLAGDWESNPCP

SEQ ID NO: 49 - protein, 58 aa, Homo sapiens
MFPTIPLSRLFDNAMLRAHRLHQLAFDTYQEFEEAYIPKEQKYSFLQNPQTSLCFSES

SEQ ID NO: 50 - protein, 58 aa, Homo sapiens
RTGQIFKQTYSKFDTNSHNDDALLKNYGLLYCFRKDMDKVETFLRIVQCRSVEGSCGF

SEQ ID NO: 51 - protein, 58 aa, Homo sapiens
IPTPSNRETQQKSNLELLRISLLIQSVYGASDSNVYDLLKDLEEGIQTLMGRLEDGSP

SEQ ID NO: 52 - protein, 134 aa, Homo sapiens
MDMRTPAQFLGILLLWFPGIKCDIKMTQSPSSMYASLGERVTFTCKASQDINNYLCWFQQKPGKSPKTLIYRANRLVDGVPSRFSGSGSGQDYSLTISSLEYEDMGIYYCLQYDEFPYTFGGGTKLEIKRADAA

SEQ ID NO: 53 - protein, 200 aa, Homo sapiens
MAAAGQLCLLYLSAGLLSRLGAAFNLDTREDNVIRKYGDPGSLFGFSLAMHWQLQPEDKRLLLVGAPRAEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDF

SEQ ID NO: 54 - protein, 119 aa, Homo sapiens
VQLQQSGAELVKPGASVKLSCTASGFNIKDTYMHWVKQRPEQGLEWIGRIDPASGDTKYDPKFQVRVTITADTSTDTAYMELSSLRSEDTAVYYCATGMWVSTGYALDFWGQGTLVTVS

SEQ ID NO: 55 - protein, 98 aa, Homo sapiens
EVQLQQSGPELVKPGDSVKMSCKASGYTFTDYYMDWVKQSHGKSLEWIGYIYPNNGGTSYNQKFKGKATLTVDKSSSTAYMELHSLTSEDSAVYYCAR

SEQ ID NO: 56 - DNA, 19 bp, Homo sapiens
tccctatcagtgatagaga

SEQ ID NO: 57 - DNA, 260 bp, Homo sapiens
ttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgc

SEQ ID NO: 58 - DNA, 563 bp, Homo sapiens
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacaggcgcgcctggccatacatcgatacggtaccgagttggcgcgcctgggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctt

SEQ ID NO: 59 - DNA, 1072 bp, Homo sapiens
cacaggtgctaatatttcaattatagctctaaatcagtggccaaaagcgtggccccgacaaaaggcttctatgggcttgattggagtaggcacagcatctgaagtctatcaaagtgctacaattttacatcgtttaagaccagatgggcaggaagtcaccatccaacctatgattactttcattcccattaatttgtaggggagagatttgttacagcaatggggtacagaaatcaccattccctctgcaatttacagtcctgagagccaaagaatgatgaccaaaatgggatatattcctgggaaaggattaggaaaaaaatgaggacggaataacccagccgattgaggtctcagttaaatttgaccgaagaggaattggatatccttcttaggtgcggccactgttgagcctccaaagcccattgcattaaaacggaaaactccaaaacctgtttgggttgatcagtggccgcttcctcaacaaaaactggaggcattacattcattggcaaaagaacaattagacaaaggacatattgaaccatctttttcaccatggaacgccccagtttttgtaattcagaaaaaattcgctagatggcgcatattgaccgacttatgagcggttaacgcagccattcaaccgatgggagctctccaaccagggttgccatctccggccatgatcccaaaagaatggccgatggtaataattgatctaaaagattgtgttttttactattcctttggctcctcaggattttgagaaatttgcatttacaatacctgccatgaataacaaggaaccagctactatatatcgatggaaagttgtaccccaaggaatgttaaatagtcaaacttttgttggaaaggttattcagcttgtcagggatcagtttcctgattgctatattattcattatgttgatgacattttatgtgctgctgcaagcagggacagacggattgagtgttttgctaggctacaagaagtggtgggacttacaggtctcgctatagcaccagataaaatccatactacaccctatcattacctgggaatgag

SEQ ID NO: 60 - DNA, 2177 bp, Homo sapiens
gaaggtgatgcagtcgaagccattgtggaggagtccgaaacttttattaaaggaaaggagagaaagacttaccagagacgccgggaagggggccaggaagaagatgcctgccacttaccccagaaccagacggatgggggtgaggtggtccaggatgtcaacagcagtgtacagatggtgatgatggaacagctggaccccacccttcttcagatgaagactgaagtaatggagggcacagtggctccagaagcagaggctgctgtggacgatacccagattataactttacaggttgtaaatatggaggaacagcccataaacataggagaacttcagcttgttcaagtacctgttcctgtgactgtacctgttgctaccacttcagtagaagaacttcagggggcttatgaaaatgaagtgtctaaagagggccttgcggaaagtgaacccatgatatgccacaccctacctttgcctgaagggtttcaggtggttaaagtgggggccaatggagaggtggagacactagaacaaggggaacttccaccccaggaagatcctagttggcaaaaagacccagactatcagccaccagccaaaaaaacaaagaaaaccaaaaagagcaaactgcgttatacagaggagggcaaagatgtagatgtgtctgtctacgattttgaggaagaacagcaggagggtctgcttcagaggttaatgcagagaaagtggttggtaatatgaagcctccaaagccaacaaaaattaaaaagaaaggtgtaaagaagacattccagtgtgagctttgcagttacacgtgtccacggcgttcaaatttggatcgtcacatgaaaagccacactgatgagagaccacacaagtgccatctctgtggcagggcattcagaacagtcaccctcctgaggaatcaccttaacacacacacaggtactcgtcctcacaagtgcccagactgcgacatggcctttgtgaccagtggagaattggttcggcatcgtcgttacaaacacacccacgagaagccattcaagtgttccatgtgcgattacgccagtgtagaagtcagcaaattaaaacgtcacattcgctctcatactggagagcgtccgtttcagtgcagtttgtgcagttatgccagcagggacacatacaagctgaaaaggcacatgagaacccattcaggggaaaagccttatgaatgttatatttgtcatgctcggtttacccaaagtggtaccatgaagatgcacattttacagaagcacacagaaaatgtggccaaatttcactgtccccactgtgacacagtcatagcccgaaaaagtgatttgggtgtccacttgcgaaagcagcattcctatattgagcaaggcaagaaatgccgttactgtgatgctgtgtttcatgagcgctatgccctcatccagcatcagaagtcacacaagaatgagaagcgctttaagtgtgaccagtgtgattacgcttgtagacaggagaggcacatgatcatgcacaagcgcacccacaccggggagaagccttacgcctgcagccactgcgataagaccttccgccagaagcagcttctcgacatgcacttcaagcgctatcacgaccccaacttcgtccctgcggcttttgtctgttctaagtgtgggaaaacatttacacgtcggaataccatggcaagacatgctgataattgtgctggcccagatggcgtagagggggaaaatggaggagaaacgaagaagagtaaacgtggaagaaaaagaaagatgcgctctaagaaagaagattcctctgacagtgaaaatgctgaaccagatctggacgacaatgaggatgaggaggagcctgccgtagaaattgaacctgagccagagcctcagcctgtgaccccagccccaccacccgccaagaagcggagaggacgaccccctggcagaaccaaccagcccaaacagaaccagccaacagctatcattcaggttgaagaccagaatacaggtgcaattgagaacattatagttgaagtaaaaaaagagccagatgctgagcccgcagagggagaggaagaggaggcccagccagctgccacagatgcccccaacggagacctcacgcccgagatgatcctcagcatgatggaccgg

SEQ ID NO: 61 - protein, 8 aa, Homo sapiens
YYYNCAGG

SEQ ID NO: 62 - RNA, 8 nt, Homo sapiens
ggguragu

SEQ ID NO: 63 - DNA, 39 bp, Homo sapiens
ctgccccaagtcgcagccatggctacaggctcccggacg

SEQ ID NO: 64 - DNA, 41 bp, Homo sapiens
gcttatcatgtctggccagctagctagaagccacagctgcc

(19) United States
(12) Patent Application Publication
(10) Pub. No.: US 2025/0281641 A1
(43) Pub. Date: Sep. 11, 2025

(54) VECTORS FOR DELIVERY OF HUMAN GROWTH HORMONE GENE AND MULTIPLE THERAPEUTIC GENES INTO CENTRAL NERVOUS SYSTEM BY CROSSING BLOOD BRAIN BARRIER

(71) Applicant: USMEDIGENE INC., North Potomac, MD (US)

(72) Inventors: Norman Zhennan LAI, North Potomac, MD (US); Fred NYBERG, Uppsala (SE); Alfhild GRÖNBLADH, Uppsala (SE); Mathias HALLBERG, Uppsala (SE)

(21) Appl. No.: 18/268,496

(22) PCT Filed: Dec. 20, 2021
(86) PCT No.: PCT/US21/64415

§ 371 Date: Jun. 20, 2023

Related U.S. Application Data
(60) Provisional application No. 63/127,882, filed on Dec. 18, 2020.

(51) Int. Cl. A61K 48/00; A61K 9/00; A61K 38/27; C12N 15/86; C12N 15/88

(52) U.S. Cl. CPC A61K 48/0058; A61K 9/0019; A61K 38/27; A61K 48/0075; C12N 15/86; C12N 15/88; C12N 2750/14143; C12N 2830/008

(57) ABSTRACT

The present disclosure provides an expression vector and a method for generating the expression vector. The expression vector includes one or more inserted foreign DNA sequences, including human growth hormone (hGH) and a plurality of therapeutic genes, where the one or more inserted foreign DNA sequences are fused with intracellular or extracellular secretion sequences; a promoter, configured to drive expression of the one or more inserted foreign DNA sequences; an insulator, configured for stable and safety expression of the one or more inserted foreign DNA sequences; a marker gene, configured to encoding one or more corresponding proteins to indicate the expression of the one or more inserted foreign DNA sequences; and a regulator element, configured to control expression of the hGH and the plurality of therapeutic genes under a controllable condition.

### CROSS-REFERENCE TO RELATED APPLICATION

This application claims the priority of U.S. Provisional Application No. 63/127,882, filed on Dec. 18, 2020, the content of which is incorporated herein by reference in its entirety.

### FIELD OF THE DISCLOSURE

The present disclosure generally relates to the field of gene delivery technology and, more particularly, relates to vectors for delivery of human growth hormone gene and multiple therapeutic genes into a central nervous system (CNS) by crossing a blood brain barrier.

### BACKGROUND

Growth hormone (GH) replacement therapy may improve mental disabilities in patients with adult GH deficiency and childhood-onset GH deficiency. In addition to normalizing growth and metabolism in adult patients with GH deficiency, the hormone therapy may enhance energy, motivation, and well-being, and may also improve memory and cognitive capacity. Neurons are considered to play an important role on brain memory and cognitive function. GH receptors for hormone targeting are present in the brain or central nervous system of human and animals. GH is known to mediate its effects through insulin-like growth factor-1 (IGF-1), and brain receptors for this mediator have also been demonstrated in areas associated with cognitive behaviors.

GH replacement therapy in patients who are suffered from GH deficiency (GHD) may reduce cognitive problem, improve memory, increase overall life quality, and may also show neuroprotection, learning and memory improvement, and promote growth in the pre-clinical study on animal models. Gene delivery of GH or related therapeutic proteins in target cells by vectors may be the most efficient treatment for various conditions.

Although human growth hormone therapy (hGH) is safe and effective under the range of applications approved by FDA (food and drug administration), it may like other protein therapies in long-term applications that may trigger immunoreactions due to different ages or populations. It has been reported that, for the treatment of hGH on 8 GHD children with ages from 1 to 17 for 16 months, B cells percentage was transiently decreased to subnormal levels in 7 of 8 patients. T helper/suppressor ratios were decreased in all patients to subnormal values in 7 of 8 patients; and mitogen responses were decreased to below normal in all patients. Generally, the immune response to protein-based therapeutics involves T-cells, which may help activate B cells to produce antibodies including those blocking protein therapeutics. The immune response may happen if the natural protein made by the body is defective to some extent. There is a need to develop a new construction for a specific delivery system to minimize above-mentioned side effects.

### BRIEF SUMMARY OF THE DISCLOSURE

One aspect of the present disclosure provides expression vector for amplified expression in mammalian cells. The vector includes one or more inserted foreign DNA sequences, including human growth hormone (hGH) and a plurality of therapeutic genes, where the one or more inserted foreign DNA sequences are fused with intracellular or extracellular secretion sequences; a promoter, configured to drive expression of the one or more inserted foreign DNA sequences; an insulator, configured for stable and safety expression of the one or more inserted foreign DNA sequences; a marker gene, configured to encoding one or more corresponding proteins to indicate the expression of the one or more inserted foreign DNA sequences; and a regulator element, configured to control expression of the hGH and the plurality of therapeutic genes under a controllable condition.

Another aspect of the present disclosure provides a method for generating an expression vector for amplified expression in mammalian cells. The method includes fusing one or more inserted foreign DNA sequences with intracellular or extracellular secretion sequences, the one or more inserted foreign DNA sequences including human growth hormone (hGH) and a plurality of therapeutic genes; configuring a promoter to drive expression of the one or more inserted foreign DNA sequences; configuring an insulator for stable and safety expression of the one or more inserted foreign DNA sequences; configuring a marker gene to encoding one or more corresponding proteins to indicate the expression of the one or more inserted foreign DNA sequences; and configuring a regulator element control expression of the hGH and the plurality of therapeutic genes under a controllable condition.

Other aspects of the present disclosure can be understood by those skilled in the art in light of the description, the claims, and the drawings of the present disclosure.

### BRIEF DESCRIPTION OF THE DRAWINGS

The following drawings are merely examples for illustrative purposes according to various disclosed embodiments and are not intended to limit the scope of the present disclosure.

FIG. 1 depicts an exemplary multiple gene expression vector according to various disclosed embodiments of the present disclosure;

FIG. 2A depicts an exemplary fluorescent image of in vitro transduction of brain choroid plexus cells by recombinant (adenoviral associate virus) AAVs according to various disclosed embodiments of the present disclosure;

FIG. 2B depicts an exemplary fluorescent image of in vitro transduction of brain choroid plexus cells infected by AAV vectors without any included transgenes (as control) according to various disclosed embodiments of the present disclosure;

FIG. 3A depicts an exemplary fluorescent image of intra-hippocampal injection of recombinant AAV vectors into the hippocampus of a rat brain according to various disclosed embodiments of the present disclosure;

FIG. 3B depicts an exemplary fluorescent image of intra-hippocampal injection of AAV vectors without any transgenes (as control) into the hippocampus of a rat brain according to various disclosed embodiments of the present disclosure;

FIG. 3C depicts an enlarge view of a boxed region (shows various CNS cells transduced by the transgenes from recombinant AAVs delivery) in FIG. 3A;

FIG. 4A depicts an exemplary fluorescent image from the expression of transgenes (fluorescent light) in CNS tissues after tail-vein injection of recombinant AAV vectors encoding transgenes of GFP and/or human growth hormone hGH in animal models according to various disclosed embodiments of the present disclosure;

FIG. 4B depicts an exemplary fluorescent image in a control animal model after tail-vein injection of recombinant AAV vectors without any transgenes according to various disclosed embodiments of the present disclosure;

FIG. 5A depicts an exemplary quantitative-polymerase chain reaction (qPCR) detection result of mRNA expressional levels of human growth hormone hGH after the tail-vein injection according to various disclosed embodiments of the present disclosure;

FIG. 5B depicts an exemplary quantitative-PCR (qPCR) detection result of mRNA expressional levels of human growth hormone hGH after intra-hippocampal injection according to various disclosed embodiments of the present disclosure;

FIG. 6A depicts an exemplary quantitative-PCR (qPCR) detection result of mRNA expressional levels of rat IGF-1 after the tail-vein injection according to various disclosed embodiments of the present disclosure;

FIG. 6B depicts an exemplary quantitative-PCR (qPCR) detection result of mRNA expressional levels of rat IGF-1 after intra-hippocampal injection according to various disclosed embodiments of the present disclosure;

FIG. 7A depicts an exemplary fluorescent image of neurons detected by containing a specific anti-NeuN antibody with IgG conjugated to TRITC (A594/red fluorescent light, 20×), where the slide was sectioned from CNS samples after the tail-vein injection, according to various disclosed embodiments of the present disclosure;

FIG. 7B depicts an exemplary fluorescent image for co-expression of hGH in the neurons by using a specific anti-human growth hormone antibody with IgG conjugated to FITC (A488/green fluorescent light, 20×), where the slide was sectioned from CNS samples after the tail-vein injection, according to various disclosed embodiments of the present disclosure;

FIG. 8A depicts an exemplary fluorescent image of neurons for a specific neural biomarker protein which was expressed in neurons of the CNS and detected by anti-NeuN antibody (white arrows indicate red fluorescent light), where the slide was sectioned from the CNS samples after the tail-vein injection, according to various disclosed embodiments of the present disclosure;

FIG. 8B depicts an exemplary fluorescent image for the expression of hGH proteins in the cytoplasm of the neurons, detected by a specific anti-human growth hormone antibody (white arrows indicate green fluorescent staining/light), where the slide was sectioned from the CNS samples after the tail-vein injection, according to various disclosed embodiments of the present disclosure;

FIG. 8C depicts an exemplary overlapped view of FIG. 8A and FIG. 8B;

FIG. 9A depicts an exemplary fluorescent image for the expression of hGH proteins detected by a specific antibody (green fluorescent staining/light) in CNS choroid plexus, neurons identified by a specific anti-NeuN antibody (red fluorescent light), and overlapped co-localizations (yellow fluorescent light) of both hGH proteins and neurons in a same view of the tissues with the delivery of recombinant AAV vectors after tail-vein injection according to various disclosed embodiments of the present disclosure;

FIG. 9B depicts an exemplary fluorescent image for a control group without hGH protein expression according to various disclosed embodiments of the present disclosure;

FIG. 9C depicts an exemplary fluorescent image for the expression of hGH proteins in another section of CNS choroid plexus tissue with the delivery of recombinant AAV vectors after tail-vein injection according to various disclosed embodiments of the present disclosure;

FIG. 10A depicts effect of hGH treatment in tail-vein injection on body weights according to various disclosed embodiments of the present disclosure; and

FIG. 10B depicts effect of hGH treatment in CNS injection on body weights according to various disclosed embodiments of the present disclosure.

### DETAILED DESCRIPTION

References may be made in detail to exemplary embodiments of the disclosure, which may be illustrated in the accompanying drawings. Wherever possible, same reference numbers may be used throughout the accompanying drawings to refer to same or similar parts.

A cloning vector, widely used in molecular biology, is a small piece of DNA molecule, which can be inserted with a foreign DNA fragment and used as a vehicle to transfer foreign genetic material into another cell. Insertion of the foreign DNA fragment into the cloning vector may be normally carried out by digesting both the vector and the foreign DNA with a restriction enzyme and ligating the fragments digested by the restriction enzyme together. The vector may be used for controlled expression of a particular gene with specific promoter sequences such as human neuron synapsin, neuron enolase (NSE), or neuron type-specific expression (CaM kinase II) to drive transcription of the transgene cloned in the vector.

Once the vector is inside a cell, the protein that is encoded by the transgene may be produced by cellular transcription and translation. After expression of the gene product, the resulted protein of interest may need to be purified and isolated from other proteins of the host cell. To facilitate the process, the cloned gene may normally a tag sequence, such as attached histidine (His) tag. In addition, GFP (green fluorescent protein) or other human antigen sequences may often be used as markers to follow the expression process for selection. In the cells where the tagged gene is expressed, the GFP may also be produced, and those cells may be observed under fluorescence microscopy and isolated by FACS (fluorescence-activated cell sorting).

However, overcoming the difficulty of delivering therapeutic agents to specific regions of the brain presents a major challenge to the treatment of most brain disorders. In brain's neuroprotective role, the blood brain barrier (BBB) may function to hinder the delivery of various potentially important diagnostic and therapeutic agents to the brain. Therapeutic molecules and antibodies that might otherwise be effective in diagnosis and therapy do not cross the BBB in effective amounts. The blood cerebrospinal fluid barrier, which is also a function of the choroidal cells of the choroid plexus and forms the blood retinal barrier, may be considered a part of the whole realm of those barriers. For most CNS therapeutics on the market, less than 0.2% of the peripheral dose may be taken up by the brain. For example, only about 0.02% of the peripherally administered dose of morphine may enter the brain in order to produce sufficient analgesia via the CNS, but the remaining dosage in peripheral system may be more than 200 times higher than that in the brain.

According to various embodiments in the specification, an objective of the present disclosure is to deliver recombinant GH gene and/or other proteins (e.g., peptides) by a vector via crossing the BBB into neurons of the CNS to elicit therapeutic effect on patients who are suffered from neurodegenerative disorders such as intrauterine growth restriction, Alzheimer's disease, Parkinson disease, multiple sclerosis, Gaucher type-II or III disorders, neuropathic pain, spinal-cord injured, brain tumors, and the like, thereby promoting new neuron growth to recover biological functions of damaged neurons in clinical applications. Heterogonous genes or proteins used in the present disclosure may include, but not be limited to, IGF-1 (insulin growth factor-1), insulin, rat growth hormone, glucocerebrosidease, brain-derived neurotrophic factor (BDNF), β-endorphin, HSV-1 (herpes simplex virus), anti-glioma, ApoE4, presenilin 1 and/or 2, myelin oligodendrocyte glycoprotein (MOG), crry, gata4, glutamate decarboxylase 65, interleukin-10, chondroitinase ABC, P/NK-1R, human interleukin-12 (hIL-12), stem cell transcription factors for iPSC including October 3/4, Sox2, Klf1-5, Nanog, LIN28 and GIis1, and direct reprogramming genes: dopaminergic neurons reprogramming genes including ASCl1, LMX1A, MYT1L and BRN2; direct glutamatergic, GABAergic neuronal reprogramming including ASCl1, MYT1L, BRN2 and NeuroD1; direct retinal pigment epithelium reprogramming including PAX6, RAX, CRX, MITF-A, OTX2, NRL and KLF4/2; direct spinal motor neurons reprogramming including NGN2, SOX11, ISL1 and LHX3; anti-Tau, anti-β-amyloid antibodies, nanobodies (VHH), and/or any other suitable genes or proteins.

The construction of the vector encoding with hGH herein is able to cross the BBB, which may be further developed as a universal delivery system to carry any other suitable gene products to target the diseases in the CNS. The carriers may be viral gene vectors such as AAV and HIV, fusion protein or activated domain of polypeptides or peptides because the human growth (hGH) receptor is found in the human choroid plexus, which may indicate that hGH may enter the CNS via binding to its receptor on the choroid plexus tissue. The binding domain of such sequence may be selected and related sequences with capability to bind on the receptors of the BBB and may be inserted or constructed on the surfaces of the envelope proteins or capsulate proteins of the vector particles (shown in Table 1). Table 1 lists the sequences to be constructed on the surfaces of the envelopes and/or capsules of vector particles in the present disclosure. In addition, expression or production of fusion protein with capability to cross the blood brain barrier may be designed and realized in the vector construct using the vector system, which is potentially useful for the treatment of patients with CNS disorders and other diseases. The one or more inserted foreign DNA sequences may be fused with fusion sequences, which may be intracellular or extracellular secretion sequences. For example, the fusion sequences may include ROIKIWFONRR, YGRKKRRORRR, and/or LLNFDLLKILLAGDWESNPCP, which may be fused with hGH and other therapeutic genes in the rAAV vector.

TABLE 1

| The sequences to be | |
|---|---|
| constructed on the | |
| surfaces of the envelopes | |
| and/or capsules of vector | |
| particles in the present | Sequence |
| disclosure | names: |
| MFPTIPLSRLFDNAMLRAHRLHQLAF | Human growth |
| DTYQEFEEAYIPKEQKYSFLQNPQTS | hormone |
| LCFSES | receptor (hGHR) |
| | binding |
| | sequence 1 |
| RTGQIFKQTYSKFDTNSHNDDALLKN | hGHR binding |
| YGLLYCFRKDMDKVETFLRIVQCRSV | sequence 2 |
| EGSCGF | |
| IPTPSNRETQQKSNLELLRISLLIQS | hGHR binding |
| VYGASDSNVYDLLKDLEEGIQTLMGR | sequence 3 |
| LEDGSP | |
| MDMRTPAQFLGILLLWFPGIKCDIKM | The sequences |
| TQSPSSMYASLGERVTFTCKASQDIN | designed |
| NYLCWFQQKPGKSPKTLIYRANRLVD | to bind on |
| GVPSRFSGSGSGQDYSLTISSLEYED | the human |
| MGIYYCLQYDEFPYTFGGGTKLEIKR | transferrin |
| ADAA | receptor |
| | in the BBB |
| MAAAGQLCLLYLSAGLLSRLGAAFNL | The antibody |
| DTREDNVIRKYGDPGSLFGFSLAMHW | sequences |
| QLQPEDKRLLLVGAPRAEALPLQRAN | designed to |
| RTGGLYSCDITARGPCTRIEFDNDAD | bind on the |
| PTSESKEDQWMGVTVQSQGPGGKVVT | Integrin α6β4 |
| CAHRYEKRQHVNTKQESRDIFGRCYV | |
| LSQNLRIEDDMDGGDWSFCDGRLRGH | |
| EKFGSCQQGVAATFTKDF | |
| VQLQQSGAELVKPGASVKLSCTASGF | The sequences |
| NIKDTYMHWVKQRPEQGLEWIGRIDP | designed |
| ASGDTKYDPKFQVRVTITADTSTDTA | to bind on |
| YMELSSLRSEDTAVYYCATGMWVSTG | Integrins |
| YALDFWGQGTLVTVS | in the BBB |
| EVQLQQSGPELVKPGDSVKMSCKASG | The sequences |
| YTFTDYYMDWVKQSHGKSLEWIGYIY | designed |
| PNNGGTSYNQKFKGKATLTVDKSSST | to bind on |
| AYMELHSLTSEDSAVYYCAR | laminin in |
| | the BBB |

Stem cell therapy may only work desirably for long-term clinical treatment of genetic modifications with encoding particularly biological functions. The stem cells transduced by the vector system encoding growth hormone gene and/or other genes (i.e., insulin, IGF-1, reprogramming factors) may be induced and differentiated into specific cell types such as the neurons being capable to secret hGH and/or express desirable therapeutic proteins which may be subsequently transplanted in the CNS of neurodegenerative patients, the islet cells secreting insulin implanted into pancreatic tissues for diabetic patients, and the bone marrow stem cells with expressing normal enzyme of glucocerebrosidase infused to the patients with Gaucher disease and the like.

A brain tumor is an intracranial solid neoplasm within the brain or the central spinal canal. Any brain tumor is inherently serious and life-threatening because of its invasive and infiltrative character in the limited space of the intracranial cavity. The treatment of such disease may be limited due to the BBB, and the vector system in the present disclosure may provide an alternative and useful platform to deliver therapeutic genes or proteins in the target tissues or cells in the CNS crossing the BBB.

The viral gene vector may be used as a useful investigative tool. For example, for an animal model of Sjorgren syndrome, an autoimmune disorder was generated by using the AAV vector encoding a gene of BMP6 (bone morphogenetic protein 6), which is up-regulated in both patients and Aec1/Aec2 knock-out mice. The AAV-BMP6 vector was delivered into the submandibular glands by retrograde instillation. For another example, the delivery of a gene encoding a beta-amyloid, a precursor protein leading neurofibrillary tangles and plaques found in Alzheimer's brain, into the cortex or hippocampus of animal brain by the vector may generate a rapid animal model of Alzhemer's disease. Such approach may be extremely helpful for rapid generation of an acute animal (rat) model, which may be a desirable pre-clinical model for behavior testing required in pharmaceutical industry.

This viral gene vector may be designed to encode and/or to express the “biological tracers” to label tissues or cells, such as (AAV) viral labeled neurons in vitro and in vivo, which may be combined with laser-captured microscope to select a single cell (neuron) or sample to perform microarray study or may monitor AMPA (α-amino-3-hydroxy-5-methyl-4-isoxazolepropionic acid) receptor (i.e. expressing GluR1-GFP fusion) trafficking during neuronal synaptic plasticity to understand the mechanism of learning, memory, and drug addiction.

In various embodiments of the present disclosure, a gene-based therapy may be developed to avoid potential immune response from a therapeutic protein in the delivery system. The vector delivery system may be genetically engineered under multiple regulator elements such as the capsule or envelope of the viral gene-vector with specific binding to target the tissues or cells where the therapeutic protein is expressed under a specific promoter such as hGH in the neurons. In one embodiment, the regulator elements may include (1) Tetracycline response element (TRE) including the sequences of “TCCCTATCAGTGATAGAGA” to be included into the vector, for example placed it in front of the promoter, in order to turn on or turn off the expression of transgenes or therapeutic genes under the condition in the present of inducer, such as by doxycycline reagent or drug; and/or (2) post-transcriptional regulatory element (PRE)+derived from the woodchuck hepatitis virus (WHV) into the 3′ untranslated region of AAV or lentiviral gene transfer vectors to enhance both titer and transgene expression. In embodiments, the regulator elements may include sequences of “ttgtcaggc aacgtggcgt ggtgtgcact gtgtttgctg acgcaacccc cactggttgg ggcattgcca ccacctgtca gctcctttcc gggacttteg ctttccccct ccctattgcc acggcggaac tcatcgccgc ctgccttgcc cgctgctgga caggggctcg gctgttgggc actgacaatt ccgtggtgtt gtcggggaag ctgacgtcct ttccatggct gctegcctgt gttgccacct ggattctgcg c”. Therefore, patients may be treated with the therapeutic protein which is less likely to cause immune response and other side effects. For example, a multiple and regulated rAAV and/or lentiviral gene-vector system may be generated.

In various embodiments of the present disclosure, a multiple viral gene vector may be constructed, where the gene vector may include mammalian cell promoters or cell specific promoters, inserted nucleotide sequences encoding hGH and therapeutic genes, and biomarkers for treating the CNS diseases.

In various embodiments of the present disclosure, a viral gene vector, including a neuronal promoter or other specific promoters that function in neuron cells and in specific tissues or specific cell types, may be constructed.

In various embodiments of the present disclosure, a method for delivery of the gene encoding a therapeutic protein and hGH into the CNS by crossing the BBB may be provided.

In various embodiments of the present disclosure, a gene-based therapy may be provided to cure neurological disorders, neuropathic pain by crossing the BBB, by inserting therapeutic gene sequences into the vector, hGH, glucocerebrosidase, beat-endorphin, antisense, shRNA, mRNA, CRISPR/Cas9, iPSC stem cell transcription factors, and tissue direct reprogramming factors.

In various embodiments of the present disclosure, a combination technique of genetic engineering stem cells transduced by the vector encoding hGH may be provided to produce long-term and stable therapeutic effect on patients with the spinal-cord injured, skin burned, diabetes, and other related disease, with the combination with cell therapy or cell transplantation.

Various embodiments of the present disclosure provide a delivery system for carrying the therapeutic genes with direct iPSC (induced pluripotent stem cells) functions such as Sox2, October 4, Nanog, Lin28, Klf2 and other related stem cell(s). Only one gene and/or a combination of multiple same or different iPSC genes may be used in the present disclosure, which may not be limited according to various embodiments of the present disclosure. For example, one gene such as SOX2 in a vector system may be used for treatment, but also two or more genes, such as SOX2 and October 4 or SOX2, Oct4, and Naong cloned in a vector system, may be used for treatment. The degeneration of neuronal or CNS cells may be converted stem cell-like cells and then subsequently differentiated back to original neurons and other CNS cells with normal functionals in the microenvironment within the CNS system and/or after crossing the BBB.

Various embodiments of the present disclosure provide a delivery system of carry the therapeutic genes with direct tissue specific reprogramming functions such as ASCl1, LMX1A, MYT1L, BRN2, NeuroD1, PAX6, RAX, CRX, MITF-A, OTX2, NRL, NGN2, SOX11, ISL1, LHX3 and other related direct reprogramming factors and/or genes. Only one gene and/or combination of multiple same or different direct tissue specific reprogramming factors or genes may be used in the present disclosure, which may not be limited according to various embodiments of the present disclosure. For example, one gene such as ASCL1 in a vector system may be used for treatment, but also two or more genes, such as ASCII and MYT1L, or MYT1L, BRN2, and NeuroD1 cloned in a vector system, may be used for treatment. The degeneration of neuronal or CNS cells may be converted to be reprogrammed and differentiated back to original neurons and other CNS cells with normal functionals in the microenvironment within the CNS system and/or after crossing the BBB.

Various embodiments of the present disclosure provide a delivery system of carry the therapeutic genes with any of combinations from the direct reprogramming factors and iPSC stem cell transcriptional factors; and/or combination of multiple same or different factors or genes. The degeneration of neuronal or CNS cells may be converted to be reprogrammed and turned into stem cell-like cells by iPSC and then subsequently differentiated back to original neurons and other CNS cells with normal functionals in the microenvironment within the CNS system and/or after crossing the BBB.

In various embodiments of the present disclosure, a delivery system, carrying specific anti-cancer genes, antibodies of full or single-chain variable fragment (scFv), nanobody of VHH (variable heavy homodimers), proteins, and reagents to treat the CNS diseases including tumors or cancers by crossing the BBB, may be provided.

Various embodiments of the present disclosure may provide a method for generating an investigative tool as “biological tracer” to study or recognize the signal interaction among cell-types and to directly deliver transgene to a particular region to produce a rapid rodent animal model (rat) for pharmaceutical research and industry.

Various embodiments of the present disclosure provide a vector for delivery of human growth hormone genes and multiple therapeutic genes into a central nervous system by crossing a blood brain barrier. The vector may be a viral vector, and also be a non-viral vector such as lipid nanoparticles (LNP), which may not be limited according to various embodiments of the present disclosure. The exemplary vector is described in detail according to the present disclosure hereinafter.

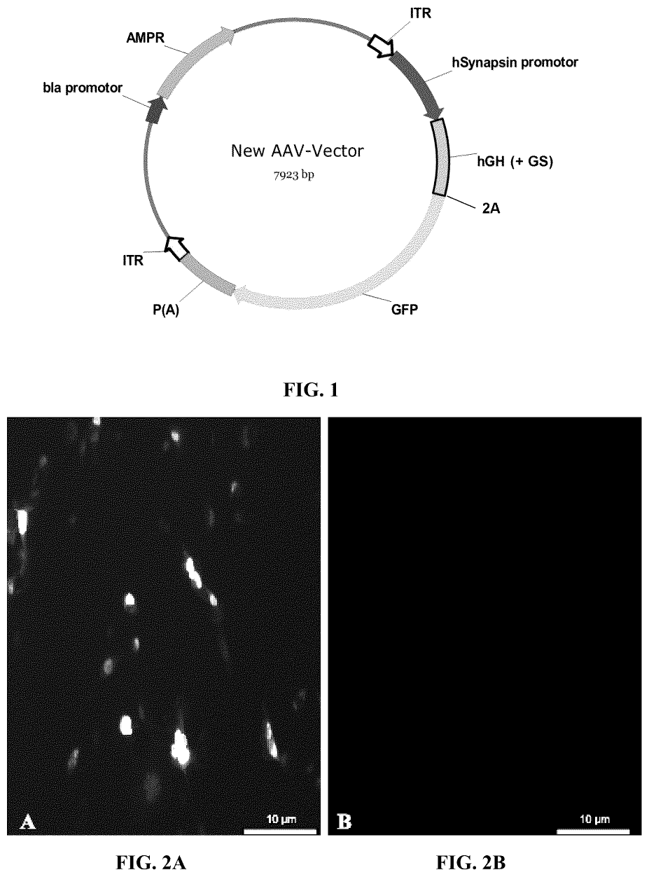

FIG. 1 depicts an exemplary multiple gene expression vector according to various disclosed embodiments of the present disclosure.

Referring to FIG. 1, the AAV multiple gene vector may be constructed with a human specific neuronal promoter of h-Synapsin followed by inserted nucleotide sequences of a cDNA encoding hGH which is linked to a growth hormone genome sequence named as “GS” and is also linked to a 2A sequence or other cleavable sequence for expression of a marker gene of GFP or Laz. In addition of such new AAV gene expression cassette, an adenovirus helper and a AAVRH 10 of Rep-Cap may be co-transfected into 293T cells to generate recombinant AAVs, which may express the hGH protein specified in the CNS neurons by crossing the BBB.

In one embodiment, additional functional sequences may be included in the present disclosure. For example, insulators such as chromatin insulators from mammalian cells may be constructed in the vectors for stable and safety expression of therapeutic genes without toxicity due to the possible integration of the vectors. Relevant sequences of the insulators may be summarized in the Table 2. Table 2 lists exemplary insulators DNA sequences used in the present disclosure. The insulator used herein may include a part or whole of the first chromatin insulator, a part or whole of the second chromatin insulator, and/or a part or whole of the third chromatin insulator. Other variant sequences with the same function as insulators, including AAV-insulator-h-Synapsin (promoter)-therapeutic gene (hGH)-insulator, may not be limited according to various embodiments of the present disclosure.

TABLE 2

| Chromatin insulators DNA | |
|---|---|
| sequences used in the present disclosure | Names of insulators |
| GAGCTCACGGGGACAGCCCCCCCCCAAAGCCCCCAGGGA | First chromatin insulator |
| TGTAATTACGTCCCTCCCCCGCTAGGGGGCAGCAGCGAG | |
| CCGCCCGGGGCTCCGCTCCGGTCCGGCGCTCCCCCCGCA | |
| TCCCCGAGCCGGCAGCGTGCGGGGACAGCCCGGGCACGG | |
| GGAAGGTGGCACGGGATCGCTTTCCTCTGAACGCTTCTC | |
| GCTGCTCTTTGAGCCTGCAGACACCTGGGGGGATACGGG | |
| GAAAAAGCTTTAGGCTGAAAGAGAGATTTAGAATGACAG | |
| GCGCGCCTGGCCATACATCGATACGGTACCGAGTTGGCG | |
| CGCCTGGGAGCTCACGGGGACAGCCCCCCCCCAAAGCCC | |
| CCAGGGATGTAATTACGTCCCTCCCCCGCTAGGGGGCAG | |
| CAGCGAGCCGCCCGGGGCTCCGCTCCGGTCCGGCGCTCC | |
| CCCCGCATCCCCGAGCCGGCAGCGTGCGGGGACAGCCCG | |
| GGCACGGGGAAGGTGGCACGGGATCGCTTTCCTCTGAAC | |
| GCTTCTCGCTGCTCTTTGAGCCTGCAGACACCTGGGGGG | |
| ATACGGGGAAAAAGCTT | |
| CACAGGTGCTAATATTTCAATTATAGCTCTAAATCAGTGG | Second chromatin insulator |
| CCAAAAGCGTGGCCCCGACAAAAGGCTTCTATGGGCTTGA | |
| TTGGAGTAGGCACAGCATCTGAAGTCTATCAAAGTGCTAC | |
| AATTTTACATCGTTTAAGACCAGATGGGCAGGAAGTCACC | |
| ATCCAACCTATGATTACTTTCATTCCCATTAATTTGTAGG | |
| GGAGAGATTTGTTACAGCAATGGGGTACAGAAATCACCAT | |
| TCCCTCTGCAATTTACAGTCCTGAGAGCCAAAGAATGATG | |
| ACCAAAATGGGATATATTCCTGGGAAAGGATTAGGAAAAA | |
| AATGAGGACGGAATAACCCAGCCGATTGAGGTCTCAGTTA | |
| AATTTGACCGAAGAGGAATTGGATATCCTTCTTAGGTGCG | |
| GCCACTGTTGAGCCTCCAAAGCCCATTGCATTAAAACGGA | |
| AAACTCCAAAACCTGTTTGGGTTGATCAGTGGCCGCTTCC | |
| TCAACAAAAACTGGAGGCATTACATTCATTGGCAAAAGAA | |
| CAATTAGACAAAGGACATATTGAACCATCTTTTTCACCAT | |
| GGAACGCCCCAGTTTTTGTAATTCAGAAAAAATTCGCTAG | |
| ATGGCGCATATTGACCGACTTATGAGCGGTTAACGCAGCC | |
| ATTCAACCGATGGGAGCTCTCCAACCAGGGTTGCCATCTC | |
| CGGCCATGATCCCAAAAGAATGGCCGATGGTAATAATTGA | |
| TCTAAAAGATTGTGTTTTTTACTATTCCTTTGGCTCCTCA | |
| GGATTTTGAGAAATTTGCATTTACAATACCTGCCATGAAT | |
| AACAAGGAACCAGCTACTATATATCGATGGAAAGTTGTAC | |
| CCCAAGGAATGTTAAATAGTCAAACTTTTGTTGGAAAGGT | |
| TATTCAGCTTGTCAGGGATCAGTTTCCTGATTGCTATATT | |
| ATTCATTATGTTGATGACATTTTATGTGCTGCTGCAAGCA | |
| GGGACAGACGGATTGAGTGTTTTGCTAGGCTACAAGAAGT | |
| GGTGGGACTTACAGGTCTCGCTATAGCACCAGATAAAATC | |
| CATACTACACCCTATCATTACCTGGGAATGAG | |
| GAAGGTGA TGCAGTCGAA GCCATTGTGG AGGAGTCCGA | Third chromatin insulator |
| AACTTTTATTAAAGGAAAGG AGAGAAAGAC TTACCAGAGA | |
| CGCCGGGAAG GGGGCCAGGA AGAAGATGCC TGCCACTTAC | |
| CCCAGAACCAGACGGATGGG GGTGAGGTGG TCCAGGATGT | |
| CAACAGCAGT GTACAGATGG TGATGATGGA ACAGCTGGAC | |
| CCCACCCTTC TTCAGATGAA GACTGAAGTA ATGGAGGGCA | |
| CAGTGGCTCC AGAAGCAGAG GCTGCTGTGG ACGATACCCA | |
| GATTATAACT TTACAGGTTG TAAATATGGA GGAACAGCCC | |
| ATAAACATAG GAGAACTTCA GCTTGTTCAA GTACCTGTTC | |
| CTGTGACTGT ACCTGTTGCT ACCACTTCAG TAGAAGAACT | |
| TCAGGGGGCT TATGAAAATG AAGTGTCTAA AGAGGGCCTT | |
| GCGGAAAGTG AACCCATGAT ATGCCACACC CTACCTTTGC | |
| CTGAAGGGTT TCAGGTGGTT AAAGTGGGGG CCAATGGAGA | |
| GGTGGAGACA CTAGAACAAG GGGAACTTCC ACCCCAGGAA | |
| GATCCTAGTT GGCAAAAAGA CCCAGACTAT CAGCCACCAG | |
| CCAAAAAAAC AAAGAAAACCAAAAAGAGCA AACTGCGTTA | |
| TACAGAGGAG GGCAAAGATG TAGATGTGTC TGTCTACGAT | |
| TTTGAGGAAG AACAGCAGGA GGGTCTGCT TCAGAGGTTA | |
| ATGCAGAGAA AGTGGTTGGT AATATGAAGC CTCCAAAGCC | |
| AACAAAAATT AAAAAGAAAG GTGTAAAGAA GACATTCCAG | |
| TGTGAGCTTT GCAGTTACAC GTGTCCACGG CGTTCAAATT | |
| TGGATCGTCA CATGAAAAGC CACACTGATG AGAGACCACA | |
| CAAGTGCCAT CTCTGTGGCA GGGCATTCAG AACAGTCACC | |
| CTCCTGAGGA ATCACCTTAA CACACACACA GGTACTCGTC | |
| CTCACAAGTG CCCAGACTGC GACATGGCCT TTGTGACCAG | |
| TGGAGAATTG GTTCGGCATC GTCGTTACAA ACACACCCAC | |
| GAGAAGCCAT TCAAGTGTTC CATGTGCGAT TACGCCAGTG | |
| TAGAAGTCAG CAAATTAAAA CGTCACATTC GCTCTCATAC | |
| TGGAGAGCGT CCGTTTCAGT GCAGTTTGTG CAGTTATGCC | |
| AGCAGGGACA CATACAAGCT GAAAAGGCACATGAGAACCC | |
| ATTCAGGGGA AAAGCCTTAT GAATGTTATA TTTGTCATGC | |
| TCGGTTTACC CAAAGTGGTA CCATGAAGAT GCACATTTTA | |
| CAGAAGCACA CAGAAAATGT GGCCAAATTT CACTGTCCCC | |
| ACTGTGACAC AGTCATAGCC CGAAAAAGTG ATTTGGGTGT | |
| CCACTTGCGA AAGCAGCATT CCTATATTGA GCAAGGCAAG | |
| AAATGCCGTT ACTGTGATGC TGTGTTTCAT GAGCGCTATG | |
| CCCTCATCCA GCATCAGAAG TCACACAAGA ATGAGAAGCG | |
| CTTTAAGTGT GACCAGTGTG ATTACGCTTG TAGACAGGAG | |
| AGGCACATGA TCATGCACAA GCGCACCCAC ACCGGGGAGA | |
| AGCCTTACGC CTGCAGCCAC TGCGATAAGA CCTTCCGCCA | |
| GAAGCAGCTT CTCGACATGC ACTTCAAGCG CTATCACGAC | |
| CCCAACTTCG TCCCTGCGGC TTTTGTCTGT TCTAAGTGTG | |
| GGAAAACATT TACACGTCGG AATACCATGG CAAGACATGC | |
| TGATAATTGT GCTGGCCCAG ATGGCGTAGA GGGGGAAAAT | |
| GGAGGAGAAA CGAAGAAGAG TAAACGTGGA AGAAAAAGAA | |
| AGATGCGCTC TAAGAAAGAA GATTCCTCTG ACAGTGAAAA | |
| TGCTGAACCAGATCTGGACG ACAATGAGGA TGAGGAGGAG | |
| CCTGCCGTAG AAATTGAACC TGAGCCAGAGCCTCAGCCTG | |
| TGACCCCAGC CCCACCACCC GCCAAGAAGC GGAGAGGACG | |
| ACCCCCTGGCAGAACCAACC AGCCCAAACA GAACCAGCCA | |
| ACAGCTATCA TTCAGGTTGA AGACCAGAAT ACAGGTGCAA | |
| TTGAGAACAT TATAGTTGAA GTAAAAAAAG AGCCAGATGC | |
| TGAGCCCGCA GAGGGAGAGG AAGAGGAGGC CCAGCCAGCT | |
| GCCACAGATG CCCCCAACGG AGACCTCACG CCCGAGATGA | |
| TCCTCAGCAT GATGGACCGG | |

In one embodiment, specific promoters for transducing the CNS cell types, such as h-Synapsin promoter and h-GFAP promoter, may also be included.

In one embodiment, the expression cassettes (EC) of the vector of AAV, or Lentiviral vector or LNP, may also include the following sequences for enhancing transgene expression: poly Y-N-C-A-G-G (splicing acceptor sequences) at the 5′ end of the EC, and G-G-G-U-R-A-G-U (splicing donor sequences) at the 3′ end of the EC. For example, the expression cassette in the AAV vector: AAV-poly Y-N-C-A-G-G-insulator-h-Synapsin (promoter)-therapeutic genes (hGH-2A-NGN2-2A-SOX2)-insulator-G-G-G-U-R-A-G-U.

In one embodiment, in order to deliver recombinant AAV or lentiviral vectors into the CNS by crossing the BBB, envelope and capsule with genetic engineered polypeptides (as shown in Table 1) may target the receptors located on the BBB.

Exemplarily, referring to FIG. 1, the vector, designed on its application for delivery and production of the protein such as hGH in the CNS neurons in vivo and mammalian cells in vitro, may include a cytomegalovirus (CMV) promoter, a glial fibrillary acidic protein (GFAP) promoter for astrocyte in the CNS, modified nucleotide sequences, and a GFP gene. The vector may be digested by BamH-I and Hind III at 37° C. for 1.5-2 hours; the sequence of insert-1 of hGH gene may be cloned into the digested AAV-2 vector; and the sequence of insert-2 may be isolated from a pUC12 vector by digestion of Hind III and Cla I.

The sequence of cDNA of hGH may be generated by polymerase chain reaction (PCR) using the primers designed as shown in the following. Designed PCR primers for cloning human growth hormone gene into the vector with human synapsin neuron specific promoter may be illustrated herein, where the PCR primers may include a forward primer (A) and a reverse primer (B).

```
A) Forward primer (sense):
5′-CTG CCC CAA GTC GCA GCC ATG GCT ACA GGC TCC CGG ACG-3′
            Vector (hSyn) Nco-I hGH(start sequence)
B) Reverse primer (antisense):
5′-G CTT ATC ATG TCT GGC CAG CT AG CTA GAA GCC ACA GCT GCC-3′
          Vector (hSyn-Lucia) Nhe-I hGH (end sequence)
```

Exemplarily, the PCR product fragment of hGH with primers containing partial sequences that are overlapped with the neuronal vector may be cloned as a new vector B containing cDNA of hGH linked to a gene of GFP or Laz, using a Cold Fusion Protocol (System Bioscience). The vector B may then be digested by Ase I and Hind III. The fragment containing two genes expression cassettes driven by the neuronal promoter of hSynapsin may be subsequently cloned back to the region of the vector A by digestion of Not I and Cla I to generate a new vector named as GS vector.

FIG. 2A depicts an exemplary fluorescent image of in vitro transduction of brain choroid plexus cells by recombinant (adenoviral associate virus) AAVs according to various disclosed embodiments of the present disclosure; and FIG. 2B depicts an exemplary fluorescent image of in vitro transduction of brain choroid plexus cells infected by AAV vectors without any included transgenes (as the control) according to various disclosed embodiments of the present disclosure. In various embodiments, serotypes of AAV vectors may be, such as AAV1, AAV2, AAV3, AAV 4, AAV5, AAV6, AAV7, AAV8, AAV9, AAV (Rh) 10, AAV11, AAV12, etc.

Referring to FIG. 2A, it may indicate newly prepared AAVs that carry both GFP and human growth hormone. Only 1.0 μL of such viral vector with titer (concentration) of 10¹³ pfu/mL may be applied to sufficiently transduce the humane choroid plexus cells (4×10⁴) in vitro.

The new multiple gene vector may be capable of delivering at least two proteins which include a therapeutic protein (i.e., hGH) expressing in mammalian cells and another protein as a biomarker (e.g., GFP) in the cells transduced by such vector system, where the transduced cells may be selected and distinguished from un-transduced cells. In addition, these proteins may be expressed in specific cell-types and/or tissues, for example, neurons may be driven by a neuronal promoter.

According to various embodiments of the present disclosure, an expression vector for amplified expression in mammalian cells is provided. The vector includes one or more inserted foreign DNA sequences, including human growth hormone (hGH) and a plurality of therapeutic genes, where the one or more inserted foreign DNA sequences are fused with intracellular or extracellular secretion sequences; a promoter, configured to drive expression of the one or more inserted foreign DNA sequences; an insulator, configured for stable and safety expression of the one or more inserted foreign DNA sequences; a marker gene, configured to encoding one or more corresponding proteins to indicate the expression of the one or more inserted foreign DNA sequences; and a regulator element, configured to control expression of the hGH and the plurality of therapeutic genes under a controllable condition.

In one embodiment, the expression vector includes an adenoviral associate virus (AAV) vector and/or a lentiviral vector (e.g., including a human immunodeficiency virus (HIV) vector), and/or a non-viral vector (e.g., including a lipid nanoparticle (LNP) vector).

In one embodiment, the promoter includes a cytomegalovirus (CMV) promoter or an elongation factor (EF-1) promoter or a synthetic specific promoter.

In one embodiment, the marker gene includes a green fluorescent protein (GFP) gene and/or a non-toxic mammalian biomarker.

In one embodiment, the expression vector further includes a neuronal promoter, including human synapsin, configured to drive expression of transgenes in neurons in a central nervous system (CNS).

In one embodiment, the insulator includes one or more of a first chromatin insulator, a second chromatin insulator, and a third chromatin insulator.

The first chromatin insulator includes at least a part of a sequence of:

GAGCTCACGGGGACAGCCCCCCCCCAAAGCCCCCAGGGA
TGTAATTACGTCCCTCCCCCGCTAGGGGGCAGCAGCGAG
CCGCCCGGGGCTCCGCTCCGGTCCGGCGCTCCCCCCGCA
TCCCCGAGCCGGCAGCGTGCGGGGACAGCCCGGGCACGG
GGAAGGTGGCACGGGATCGCTTTCCTCTGAACGCTTCTC
GCTGCTCTTTGAGCCTGCAGACACCTGGGGGGATACGGG
GAAAAAGCTTTAGGCTGAAAGAGAGATTTAGAATGACAG
GCGCGCCTGGCCATACATCGATACGGTACCGAGTTGGCG
CGCCTGGGAGCTCACGGGGACAGCCCCCCCCCAAAGCCC
CCAGGGATGTAATTACGTCCCTCCCCCGCTAGGGGGCAG
CAGCGAGCCGCCCGGGGCTCCGCTCCGGTCCGGCGCTCC
CCCCGCATCCCCGAGCCGGCAGCGTGCGGGGACAGCCCG
GGCACGGGGAAGGTGGCACGGGATCGCTTTCCTCTGAAC
GCTTCTCGCTGCTCTTTGAGCCTGCAGACACCTGGGGGG
ATACGGGGAAAAAGCTT;

The second chromatin insulator includes at least a part of a sequence of:

CACAGGTGCTAATATTTCAATTATAGCTCTAAATCAGTGG
CCAAAAGCGTGGCCCCGACAAAAGGCTTCTATGGGCTTGA
TTGGAGTAGGCACAGCATCTGAAGTCTATCAAAGTGCTAC
AATTTTACATCGTTTAAGACCAGATGGGCAGGAAGTCACC
ATCCAACCTATGATTACTTTCATTCCCATTAATTTGTAGG
GGAGAGATTTGTTACAGCAATGGGGTACAGAAATCACCAT
TCCCTCTGCAATTTACAGTCCTGAGAGCCAAAGAATGATG
ACCAAAATGGGATATATTCCTGGGAAAGGATTAGGAAAAA
AATGAGGACGGAATAACCCAGCCGATTGAGGTCTCAGTTA
AATTTGACCGAAGAGGAATTGGATATCCTTCTTAGGTGCG
GCCACTGTTGAGCCTCCAAAGCCCATTGCATTAAAACGGA
AAACTCCAAAACCTGTTTGGGTTGATCAGTGGCCGCTTCC
TCAACAAAAACTGGAGGCATTACATTCATTGGCAAAAGAA
CAATTAGACAAAGGACATATTGAACCATCTTTTTCACCAT
GGAACGCCCCAGTTTTTGTAATTCAGAAAAAATTCGCTAG
ATGGCGCATATTGACCGACTTATGAGCGGTTAACGCAGCC
ATTCAACCGATGGGAGCTCTCCAACCAGGGTTGCCATCTC
CGGCCATGATCCCAAAAGAATGGCCGATGGTAATAATTGA
TCTAAAAGATTGTGTTTTTTACTATTCCTTTGGCTCCTCA
GGATTTTGAGAAATTTGCATTTACAATACCTGCCATGAAT
AACAAGGAACCAGCTACTATATATCGATGGAAAGTTGTAC
CCCAAGGAATGTTAAATAGTCAAACTTTTGTTGGAAAGGT
TATTCAGCTTGTCAGGGATCAGTTTCCTGATTGCTATATT
ATTCATTATGTTGATGACATTTTATGTGCTGCTGCAAGCA
GGGACAGACGGATTGAGTGTTTTGCTAGGCTACAAGAAGT
GGTGGGACTTACAGGTCTCGCTATAGCACCAGATAAAATC
CATACTACACCCTATCATTACCTGGGAATGAG.

The third chromatin insulator includes at least a part of a sequence of:

GAAGGTGA TGCAGTCGAA GCCATTGTGG AGGAGTCCGA
AACTTTTATTAAAGGAAAGG AGAGAAAGAC TTACCAGAGA CGCCGGGAAG
GGGGCCAGGA AGAAGATGCC TGCCACTTAC CCCAGAACCAGACGGATGGG
GGTGAGGTGG TCCAGGATGT CAACAGCAGT GTACAGATGG TGATGATGGA
ACAGCTGGAC CCCACCCTTC TTCAGATGAA GACTGAAGTA ATGGAGGGCA
CAGTGGCTCC AGAAGCAGAG GCTGCTGTGG ACGATACCCA GATTATAACT
TTACAGGTTG TAAATATGGA GGAACAGCCC ATAAACATAG GAGAACTTCA
GCTTGTTCAA GTACCTGTTC CTGTGACTGT ACCTGTTGCT ACCACTTCAG
TAGAAGAACT TCAGGGGGCT TATGAAAATG AAGTGTCTAA AGAGGGCCTT
GCGGAAAGTG AACCCATGAT ATGCCACACC CTACCTTTGC CTGAAGGGTT
TCAGGTGGTT AAAGTGGGGG CCAATGGAGA GGTGGAGACA CTAGAACAAG
GGGAACTTCC ACCCCAGGAA GATCCTAGTT GGCAAAAAGA CCCAGACTAT
CAGCCACCAG CCAAAAAAAC AAAGAAAACCAAAAAGAGCA AACTGCGTTA
TACAGAGGAG GGCAAAGATG TAGATGTGTC TGTCTACGAT TTTGAGGAAG
AACAGCAGGA GGGTCTGCT TCAGAGGTTA ATGCAGAGAA AGTGGTTGGT
AATATGAAGC CTCCAAAGCC AACAAAAATT AAAAAGAAAG GTGTAAAGAA
GACATTCCAG TGTGAGCTTT GCAGTTACAC GTGTCCACGG CGTTCAAATT
TGGATCGTCA CATGAAAAGC CACACTGATG AGAGACCACA CAAGTGCCAT
CTCTGTGGCA GGGCATTCAG AACAGTCACC CTCCTGAGGA ATCACCTTAA
CACACACACA GGTACTCGTC CTCACAAGTG CCCAGACTGC GACATGGCCT
TTGTGACCAG TGGAGAATTG GTTCGGCATC GTCGTTACAA ACACACCCAC
GAGAAGCCAT TCAAGTGTTC CATGTGCGAT TACGCCAGTG TAGAAGTCAG
CAAATTAAAA CGTCACATTC GCTCTCATAC TGGAGAGCGT CCGTTTCAGT
GCAGTTTGTG CAGTTATGCC AGCAGGGACA CATACAAGCT
GAAAAGGCACATGAGAACCC ATTCAGGGGA AAAGCCTTAT GAATGTTATA
TTTGTCATGC TCGGTTTACC CAAAGTGGTA CCATGAAGAT GCACATTTTA
CAGAAGCACA CAGAAAATGT GGCCAAATTT CACTGTCCCC ACTGTGACAC
AGTCATAGCC CGAAAAAGTG ATTTGGGTGT CCACTTGCGA AAGCAGCATT
CCTATATTGA GCAAGGCAAG AAATGCCGTT ACTGTGATGC TGTGTTTCAT
GAGCGCTATG CCCTCATCCA GCATCAGAAG TCACACAAGA ATGAGAAGCG
CTTTAAGTGT GACCAGTGTG ATTACGCTTG TAGACAGGAG AGGCACATGA
TCATGCACAA GCGCACCCAC ACCGGGGAGA AGCCTTACGC CTGCAGCCAC
TGCGATAAGA CCTTCCGCCA GAAGCAGCTT CTCGACATGC ACTTCAAGCG
CTATCACGAC CCCAACTTCG TCCCTGCGGC TTTTGTCTGT TCTAAGTGTG
GGAAAACATT TACACGTCGG AATACCATGG CAAGACATGC TGATAATTGT
GCTGGCCCAG ATGGCGTAGA GGGGGAAAAT GGAGGAGAAA CGAAGAAGAG
TAAACGTGGA AGAAAAAGAA AGATGCGCTC TAAGAAAGAA GATTCCTCTG
ACAGTGAAAA TGCTGAACCAGATCTGGACG ACAATGAGGA TGAGGAGGAG
CCTGCCGTAG AAATTGAACC TGAGCCAGAGCCTCAGCCTG TGACCCCAGC
CCCACCACCC GCCAAGAAGC GGAGAGGACG ACCCCCTGGCAGAACCAACC
AGCCCAAACA GAACCAGCCA ACAGCTATCA TTCAGGTTGA AGACCAGAAT
ACAGGTGCAA TTGAGAACAT TATAGTTGAA GTAAAAAAAG AGCCAGATGC
TGAGCCCGCA GAGGGAGAGG AAGAGGAGGC CCAGCCAGCT GCCACAGATG
CCCCCAACGG AGACCTCACG CCCGAGATGA TCCTCAGCAT GATGGACCGG

In one embodiment, a surface capsule of the expression vector is engineered by inserting a nucleotide sequence to bind and/or recognize one or more receptors on Choroid plexus and blood brain barrier (BBB) tissues.

In one embodiment, a surface capsule of the expression vector is engineered by inserting a single-chain fragment variable (scFv) antibody sequence to bind and/or recognize one or more receptors on Choroid plexus and blood brain barrier (BBB) tissues.

In one embodiment, a surface capsule of the expression vector is engineered by inserting a ligand sequence derived from the hGH to bind and/or recognize one or more hGH receptors on Choroid plexus and blood brain barrier (BBB) tissues.

In one embodiment, a biological tracer is used as an investigative and/or diagnostic tool to mark cells and/or to generate a specific animal model in vivo, and/or the biological tracer is applied for diagnostic instruments.

In one embodiment, the one or more inserted foreign DNA sequences are genes encoding proteins, peptides and transcription factors including IGF-1 (insulin growth factor-1), insulin, rat growth hormone, glucocerebrosidease, brain-derived neurotrophic factor (BDNF), β-endorphin, HSV-1 (herpes simplex virus), anti-glioma, ApoE4, presenilin 1 and/or 2, myelin oligodendrocyte glycoprotein (MOG), crry, gata4, glutamate decarboxylase 65, interleukin-10, chondroitinase ABC, P/NK-1R, human interleukin-12 (hIL-12), stem cell transcription factors for iPSC including October 3/4, Sox2, Klf1-5, Nanog, LIN28 and GIis1, direct dopaminergic neurons reprogramming including ASCl1, LMX1A, MYT1L and BRN2, direct glutamatergic, GABAergic neuronal reprogramming including ASCl1, MYT1L, BRN2. and NeuroD1, direct retinal pigment epithelium reprogramming including PAX6, RAX, CRX, MITF-A, OTX2, NRL and KLF4/2, and direct spinal motor neurons reprogramming including NGN2, SOX11, ISL1 and LHX3, and further including anti-Tau, anti-β-amyloid antibodies, and nanobodies (VHH).

In one embodiment, the one or more inserted foreign DNA sequences include CRISPR/Cas9, antisense, sense, or mRNA used for treating a central nervous system (CNS) disease.

In one embodiment, the one or more inserted foreign DNA sequences include antisense or sense used for treating a central nervous system (CNS) disease, the CNS disease including brain viral infection.

In one embodiment, the one or more inserted foreign DNA sequences include shRNA, RNAi, or mRNA used for treating a central nervous system (CNS) disease, the CNS disease including brain viral infection.

In one embodiment, the expression vector is used to treat a central nervous system (CNS) disease including brain viral infection by delivering the hGH and the plurality of therapeutic genes via crossing the BBB.

In one embodiment, delivering the hGH and the plurality of therapeutic genes into the CNS via crossing the BBB is administrated with intravenous injection formulation, nasal spray formulation, inhalation formulation, liquid and/or tablet formulations, eye drop administration formulation, or a combination thereof.

In one embodiment, the regulator element includes (1) a tetracycline response element (TRE) including sequences of “TCCCTATCAGTGATAGAGA” to be included into the vector; and/or (2) a post-transcriptional regulatory element (PRE)+derived from woodchuck hepatitis virus (WHV) into 3′ untranslated region of the AAV vector or lentiviral vector to enhance both titer and transgene expression.

In one embodiment, the intracellular or extracellular secretion sequences include fusion sequences, the fusion sequences including ROIKIWFONRR, YGRKKRRORRR, and/or LLNFDLLKILLAGDWESNPCP.

According to various embodiments of the present disclosure, a method for generating an expression vector for amplified expression in mammalian cells is provided. The method includes fusing one or more inserted foreign DNA sequences with intracellular or extracellular secretion sequences, the one or more inserted foreign DNA sequences including human growth hormone (hGH) and a plurality of therapeutic genes; configuring a promoter to drive expression of the one or more inserted foreign DNA sequences; configuring an insulator for stable and safety expression of the one or more inserted foreign DNA sequences; configuring a marker gene to encoding one or more corresponding proteins to indicate the expression of the one or more inserted foreign DNA sequences; and configuring a regulator element control expression of the hGH and the plurality of therapeutic genes under a controllable condition.

Various embodiments of the present disclosure provide detailed description of adeno-associated vector construction, cell culture and transformation, AAV production and purification, lysing of 293 cells and harvesting of rAAVs, purification of AAVs by ultracentrifugation, dialysis for in vivo application, heparin column purification of rAAVs, animal study, delivery of hGH gene into rat CNS by using the AAVs, brain immunofluorescence assay, immunocytochemical detection of cells, RNA extraction, construction of cDNA libraries, gene expression profiling with real-time PCR, protein sample preparation, hGH ELISA assay, and statistical analysis hereinafter.

In an exemplary experiment, for constructing adeno-associated vector encoding hGH and GFP, the cDNA of human growth hormone (hGH) with 598 bp was cloned into an AAV-2 vector (Applied Biological Materials Inc/abm) following enzymatic digestion by BamH-I and Hind III at 37° C. for 2 hours (New England Biolab). The sequence of GFP was isolated from pUC12 vector (Clontech) by digestion of Hind III and Cla I, and then subcloned into the AAV-2 vector (abm). The backbone of this new construct may contain a CMV promoter, a polyadenylation site, and an ampicillin resistance gene.

In an exemplary experiment, for cell culture and transformation, human brain choroid plexus, HeLa and COS-7 cell lines were obtained from the American Type Culture Collection. COS-7 and HeLa cells were grown in DMEM (GIBCO) containing 10% heat-inactivated FBS. Human endothelial cells were grown in F12 K-medium with 2 mM L-glutamine containing 1.5 g/L sodium bicarbonate, 100 mg/mL heparin, 30 mg/mL endothelial cell growth supplement (ICN), and 10% FBS. The brain choroid plexus cells were grown in

Eagle's minimum essential medium with 0.1 mM nonessential amino acids, 90% Earle's balanced salt solution, and 10% FBS. H9 cells were grown in 80% RPMI 1640 medium (GIBCO) containing 10% FBS, 1 mM L-glutamine, 0.05 mg/mL gentamicin and 10,000 units/mL Penstrep (GIBCO). Cells were infected in DMEM/FBS containing 4 mg/mL Polybrene for 4-16 h. Human Embryonic Kidney HEK293 cells were cultured and maintained in Dulbecco's modified Eagle's medium (DMEM) supplemented with 10% fetal bovine serum at 37° C. in a fully humidified atmosphere of 5% CO2. The cell culture condition may be modified based on actual needs, which may not be limited according to various embodiments of the present disclosure.

In an exemplary experiment, lipofectamine (Invitrogen) transfection kit was used for transfection Protocol. Other transfection kits or manners, commercial or noncommercial, may also be used based on actual needs, which may not be limited according to various embodiments of the present disclosure.

In an exemplary experiment, for adherent cells, 0.5-2×10⁵ cells were plated in 500 μL of growth medium without antibiotics one day before transfection, such that cells may be 90% confluent at the time of transfection.

In an exemplary experiment, for suspension cells, 4-8 x105 cells were plated in 500 μL of growth medium without antibiotics prior to preparing complexes.

In an exemplary experiment, for each transfection sample, complexes were prepared as the following. At step A, DNA was diluted in 50 μL of Opti-MEM® I Reduced Serum Medium without serum (or other medium without serum) and the mixture was mixed gently. At step B, Lipofectamine™ 2000 was gently mixed before use, then an appropriate amount was diluted in 50 μL of Opti-MEM® I Medium, which was incubated for 5 minutes at room temperature. Step C was proceeded within 25 minutes. At step C, after the 5-minute incubation, the diluted DNA was combined with diluted Lipofectamine™ 2000 (total volume is 100 μL). The mixture was mix gently and incubated for 20 minutes at room temperature (solution may appear cloudy). Complexes were stable for 6 hours at room temperature.

100 μL of complexes was added to each well containing cells and medium and mixed gently by rocking the plate back and forth. Cells were incubated at 37° C. in a CO₂ incubator for 18-48 hours prior to transgene expression test. Medium may be changed after 4-6 hours.

In an exemplary experiment, for AAV production and purification, in the preparation of human embryonic kidney 293 cell and transfection by the AAV vector, 2×106 cells were plated in ten culture plates (15 cm), and cells may be 70-80% confluent before transfection (approximately 24 hours after plating). The cells were cultured in 20 mL of standard Dulbecco's modified Eagle medium (DMEM) with low glucose containing (10%) fetal calf serum and 100 μg/mL penicillin/100 μg/mL streptomycin. 3 hours before transfection, DMEM was removed and replaced with medium containing 5% fetal calf serum. The transfection reaction in each plate may contain 12 μg of adenovirus helper plasmid, 6 μg of the cap and rep plasmid (for example, AAVRH10), and 6 μg of the vector containing transgene of hGH (as shown in FIG. 1) by the protocol of calcium phosphate (2M CaCl₂ and Hepes buffer). After 24 hours transfection, 20 mL of fresh medium with 10% fetal calf serum were added into the dishes until viral harvests.

In an exemplary experiment, for lysing of 293 cells and harvesting of rAAVs, 48-72 hours after transfection, media was removed from cell culture plates and discarded. The cells were gently washed in warm 1× phosphate buffered saline (PBS at pH 7.4). 15 mL warm PBS was added to each plate, and the cells were gently removed with a cell scraper. The suspension was collected in 50 mL tubes. The cells were pelleted at 800×g for 10 minutes. The supernatant was discarded, and the pellet was resuspended in 11 mL of TD buffer (150 mM NaCl, 5.0 mM KCl, 0.7 mM K2HPO₄, 25.0 mM Tris pH 8.0) in a 50 mL tube. The 50 mL tube containing the resuspend solution was placed into the dry ice for 10 min, thawed for 15 min in 37° C. water bath, and vortexed for 2 min for repeating this procedure twice. A fresh solution of 10% sodium deoxycholate in dH2O was prepared, and 1.25 mL of prepared fresh solution was added to each tube for a final concentration of 0.5%. Benzonase nuclease was added to a final concentration of 50 units per mL. Cellular debris was removed by centrifuging at 3000×g (1000 rpm) for 15 mins and transferred to fresh 50 mL tube. At this point, the sample may be stored at −20° C. before continuing its purification.

In an exemplary experiment, for purification of AAVs by ultracentrifugation, a concentration of 0.55 g CsCl/mL was added into the lysate solution in the 50 mL tube; after incubation for 10 min at room temperature, 5 ul of the final solution was taken for measuring its refractive index of 1.372 by a refractometer (AR200, Reichert Inc). 10 mL of final solution was loaded on a SW41 rotor that had spun for 48 hours at 38000 rpm at room temperature. The gradient solution was collected in 0.5 mL aliquots, and its refractive index at the range of 1.371-1.372 that are expected the highest titers (i.e., AAV2 and AAV5) in each fraction was determined and collected.

In an exemplary experiment, for dialysis for in vivo application, the fractions collected from CsCl gradient centrifugation were transferred in a dialysis cassette (Slide-A-Lyzer 10K, Thermo Scientific Inc) using a 21G 1½ plastic hub syringe. The cassette containing the solution was placed in a volume of 500 mL of 0.9% NaCl under the constant stirring for 2×60 min or overnight without stirring at room temperature. After the dialysis, samples were recovered by a 21G 1½ plastic hub syringes and were then aliquoted and stored at −80° C. until use or for titer determination by Q-PCR.

In an exemplary experiment, for heparin column purification of rAAVs, the Hi Trap heparin column was set up using a peristaltic pump, so that solutions flowed through the column at 1 mL per minute without introducing air bubbles into the heparin column. The column was equilibrated with 10 mL 150 mM NaCl and 20 mM Tris at pH 8.0. The 50 mL virus solution was applied to the column by flowing through the column. The column was washed with 20 mL 100 mM NaCl and 20 mM Tris at pH 8.0. A 5 mL syringe was used to continue to wash the column with 1 mL 200 mM NaCl and 20 mM Tris at pH 8.0, followed by 1 mL 300 mM NaCl and 20 mM Tris at pH 8.0; and the flow-through was discarded. The 5 mL syringe and gentle pressure were used to elute the virus from the column by applying: 1.5 mL 400 mM NaCl and 20 mM Tris at pH 8.0; 3.0 mL 450 mM NaCl and 20 mM Tris at pH 8.0; 1.5 mL 500 mM NaCl and 20 mM Tris at pH 8.0; and the column eluate were collected in a 15 mL centrifuge tube.

In an exemplary experiment, the vector was concentrated using Amicon ultra-4 centrifugal filter units with a 100,000 molecular weight cutoff. 4 mL of column eluate was loaded into the concentrator and centrifuged at 2000×g for 2 minutes (at room temperature). Flow through was discarded, and the concentrator was reloaded with remaining virus solution with repeat centrifugation. The concentrated volume may be approximately 250 μL. If the concentrated volume is significantly more than 250 μL, the flow through was discarded, and the centrifugation was continued in one minute step until the volume is approximately 250 μL. 250 μL of PBS was added to virus for a final volume of 500 μL and removed from the concentrator. The vector was filtered through a 13 mm diameter (0.2 μm) syringe filter. The vector may be aliquoted and stored at −80° C. as needed.

In an exemplary experiment, adult rats (250 g), obtained from Taconic Farm (Ejeby, Denmark), were maintained in an animal facility of Uppsala University in a temperature and light-controlled room, with food and water available ad libitum. Before the AAVs construct injection, the rats were anesthetized prior to viral vector injections. Rats were placed in a small-animal stereotactic apparatus fitted with a rat adaptor that positioned the skull horizontally between lambda and bregma. Animal experiments were approved by the Animal Care and Use Committee of Uppsala University at Biomedical Center.

In an exemplary experiment, for delivery of hGH gene into rat's CNS by using the AAVs, rats may be divided into four groups (6 animals per group); the first and second groups may receive AAVRH10 vector only via either the CNS direct injection or tail-vein injection, respectively; and the third and fourth groups may receive a combination dosage of AAVRH10-GFP at ⅓ of the total volume, and AAVRH10-hGH at ⅔ of the total volume via either the CNS direct injection or tail-vein injection, respectively. The vectors may have the conditions as following: one vector may contain both genes of hGH and GFP in one delivery system; and two individual vectors may be the 1st vector containing hGH only and the 2nd vector containing GFP only which may be delivered at the same time. The animals were anesthetized and the head was fastened in a stereotactic apparatus. Injections of viral vectors into the mouse brain were performed at the following coordinates: for injection into the hippocampus, 4.7 mm interaural to bregma, 3.5 mm to the right of the midline, and 3.0 mm depth. Three microliters of concentrated viral vectors were loaded into an internal cannula needle (C315× 33) with cannula tubing connected to a Hamilton syringe mounted onto a microinjection pump (Harvard Apparatus, Dover, MA). The viral vector solutions were delivered at a rate of 0.5 μL/min. For tail-vein injection, 100 μL of viral vectors was used.

In an exemplary experiment, for brain Immunofluorescence assay, animals were killed 2 months after injections, and the brains were dissected as the following. The whole brain was divided as two parts, one part, as intact tissue of half brain, was frozen in the dry ice immediately, another half was dissected into different parts of brain, including frontal cortex, cortex, hippocampus, striatum, and liver. Each of these samples was further divided into two pieces for both RNA extraction and protein preparation, respectively. The brain tissues were embedded in O.C.T. (optimum cutting temperature) medium (Tissue-Tek, Miles Inc., Indianapolis, USA) and frozen in a methanol/dry ice bath. The frozen tissues were sectioned at a thickness of 15 μm per coronal section using a cryostat (Bright Instrument, Huntingdon, U.K.) at −18° C.

In an exemplary experiment, for immunocytochemical detection of cells (e.g., neurons in CNS), the slides were fixed with 4% paraformaldehyde containing 4% sucrose in PBS for 10 min, and washed three times with the PBT buffer (PBS in 1×HSS, 0.1% bovine serum albumen [BSA] and 0.2% Tween 20), then blocked with 10% goat serum for 15 min. After washing three times with the PBT buffer, slides were incubated with the primary specific antibody of human growth hormone (1:500, R&D System Inc) at 4° C. overnight. The secondary antibodies conjugated with tetramethylrhodamine isothiocyanate (TRITC) with red fluorescent light (1:800) under the mounting medium of Northern Light (R&D System Inc) were then added onto slides for 30 min at room temperature. The slides were washed with the PBT buffer and analyzed by using a Zeiss 510 (Zeiss, Inc. Thornwood, NY) fluorescent microscope.

In an exemplary experiment, for RNA extraction, samples used for the gene expression or microarray study were immediately placed in RNAlater (Qiagen) for RNA extraction. Tissue samples were homogenized with a Homogenizer (OMNI-TH International Inc). The samples were mixed using the autoclaved 0.5 mm stainless tip in a tube containing 600 μL of the QIAzol lysis reagent (Qiagen), homogenized for 2 min and then placed on ice. The total RNA was extracted with a RNeasy Mini Kit (Qiagen) according to the manufacturer's recommendations. The quality of RNA was measured using a 2100 Bioanalyzer (Agilent Technologies, Palo Alto, CA). The samples which reached a 28S/18S ribosomal RNA ratio of 1.7 with RIN score >6.5 were kept for further steps on the gene expression by Q-PCR and/or microarray. Briefly, 550 μL of RNA Nano gel was loaded into a spin filter and centrifuged at 1500 rmp for 10 min at room temperature; 65 μL of the gel was mixed with 1 μL of Nano 6000 dye and centrifuged at 13000 g for 10 min at RT; 9.0 μL of this gel-dye mixture was then loaded into 3 wells marked with G of the RNA Nano Chips (Agilent) and 5.0 μL of RNA 6000 Nano Market was loaded into all 12 sample wells, subsequently; and 1.0 μL of samples were added into each sample well. The chip was then placed horizontally in the adapter of the IKA vortexes and vortexed for 1 min before loading into the Agilent 2100 bioanalyzer.

In an exemplary experiment, for construction of cDNA libraries, the total rat RNA (500 ng) was reverse transcribed using a SuperScript (VLO™) First-Strand cDNA synthesis kit according to the manufacture instruction (Invitrogen). The reaction component included 10× SuperScript Enzyme mix and 5×VILO Reaction Mix including random primers, MgCl₂, and dNTPs, and the tubes were subjected into a PCR program of 25° C. for 10 min, 42° C. for 60 min, and 85° C. for 5 min. The final samples of 1^{st} strand cDNA were stored at −20° C. until real-time PCR is needed. Expression was further validated by QPCR using a (2×) Taqman Universal PCR Master Mix (Applied Biosystem Inc). The cDNA was diluted to have the final concentration of 1.0 ng/μL.

In an exemplary experiment, for gene expression profiling with real-time PCR, the 1^{st} strand of cDNA synthesized from total RNA was used as template for real-time PCR. The reaction was carried out on an optional tube including 10 ng of the synthesized cDNA, 10 μL of a (2×) Universal PCR Master Mix (Applied Biosystem Inc) and 1.0 μL of TaqMan Probes purchased from Applied Biosystem Inc with a total final volume of 20 μL. The real-time PCR reactions were run on the Instrument (Bio-Rad).

In an exemplary experiment, for protein sample preparation, after the tissues from rat brain and liver were dissected, dissected tissues were frozen immediately on dry ice and stored at −80° C. For preparing enzyme-linked immunoassay (ELISA) samples, the individual sample (100 ug) was collected and crushed on dry ice and placed into a 5.0 mL of pre-chilled tube with 1.0 mL of cold PBS buffer containing 10 μL of protease inhibitors (Sigma). The tissue then was homogenized at a low speed for about 20 seconds per time for 3 times and kept cool on wet ice. The suspended solution was then transferred into a 1.7 mL of microcentrifuge tube and spined at 14,000 rpm at 4° C. for 15 min. The supernatant was aliquoted and stored at −80° C. 50 μL of the solution was diluted with 1:10 before running the ELISA.

In an exemplary experiment, for the hGH ELISA assay, the samples were diluted to equal concentration and then the ELISA was run using the human growth hormone ELISA kit (R & D System Inc). Brief description, 50 μL of standard, control or sample was added in each well (96 wells). The well was covered with the adhesive strip provided and incubated for 2 hours at room temperature. Each well was aspirated and washed with 400 μL of wash-buffer for three times for a total of four washes. Complete removal of liquid at each step is essential to desirable performance. After the last wash, any remaining wash-buffer was removed by aspirating or decanting, and the plate was inverted and blotted against clean paper towels. 200 μL of GH Conjugate was added to each well, which was covered with a new adhesive strip and incubated for 2 hours at room temperature. After washing step for three times, another 200 μL of substrate solution was added to each well and incubated for 30 minutes at room temperature under the dark. At the end, 50 μL of stop solution was added into each well. The color in the wells should change from blue to yellow. The plate was ready for determining the optical density of each well within 30 minutes using a Microplate Reader (Molecular Devices, LLC) set to 450 nm.

In an exemplary experiment, statistical analyses were performed using the software Prism (Graphpad Software Inc) and Excel program (Microsoft Inc). For the ELISA assay, the computer program of SortMax Pro Software (Molecular Devices, LLC) was used to test for a significant level of enhancement of antibody responses; the Kruskal-Wallis One-Way ANOVA was performed; and P-values less than 0.05 were considered significant. For gene expression, data was analyzed by using the computer program of GeneSpring (GX 11) including pre-processing raw-data, normalize data, QC samples/entities, t-test plus Benjamin correction, clustering, annotation and access biological context. Comparisons of biological functional pathway or biomarkers were run by an IPA program (Ingenuity^{@} systems) and Metacore (GeneGo) program.

The vector may be constructed in various embodiments of the present disclosure. In order to check the ability of the constructed AVV vector to transduce brain cells, the cells derived from the choroid plexus may be used. The AAV-2 vector containing the hGH gene may be tested in vitro together with the AAV-2 vector expressing GFP. Results may demonstrate a clear and distinct expression of GFP in these cells (FIGS. 2A-2B). In the following in vivo experiment, one group of rats may be given the AAVrh10-hGH vector together with the AAVrh10-GFP vector via a tail-vein injection and the other group may receive the AAVrh10-hGH and AAVrh10-GFP vectors via an intra-hippocampal injection.

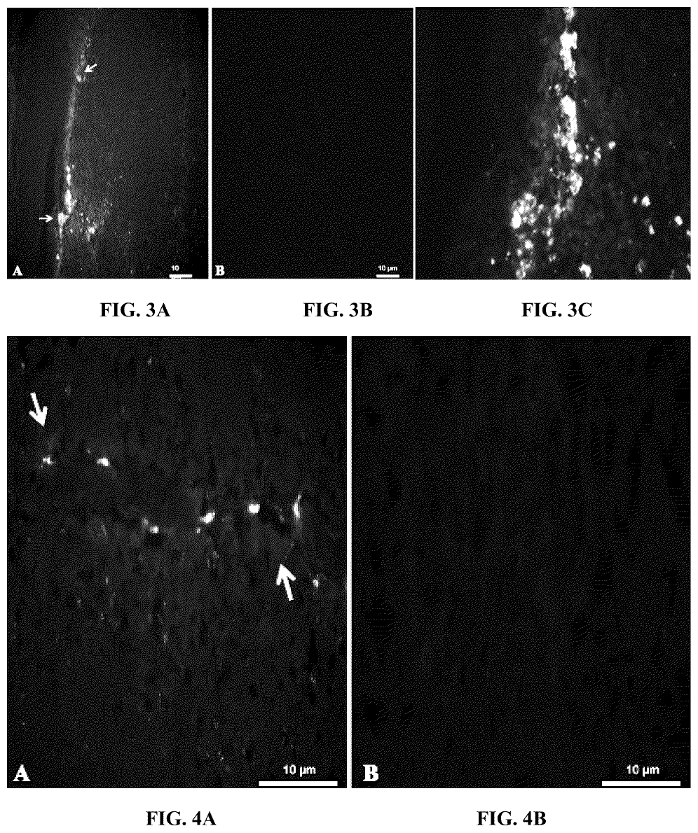

FIG. 3A depicts an exemplary fluorescent image of intra-hippocampal injection of recombinant AAV vectors into the hippocampus of a rat brain according to various disclosed embodiments of the present disclosure; FIG. 3B depicts an exemplary fluorescent image of intra-hippocampal injection of recombinant AAV vectors without any transgenes (as the control) into the hippocampus of a rat brain according to various disclosed embodiments of the present disclosure; and FIG. 3C depicts an enlarge view of a boxed region in FIG. 3A. FIG. 3A may show the expression of transgenes (fluorescent light indicated by the white arrows) in hippocampus of rat brain after in vivo intra-hippocampal injection of 5.0 μL of recombinant AAV vectors with the combination dosage of ⅔ of rAAV-hGH (3.5 μL) and ⅓ of rAAV-GFP (1.5 μL). Referring to FIG. 3B, the control group was injected with 5.0 μL AAV vector only. FIG. 3C illustrates a large view of injection site indicating significant GFP expression of transgenes in the region of hippocampus. Referring to FIG. 3C, the boxed region may show various CNS cells transduce by the transgenes from recombinant AAVs delivery. The slides were analyzed using a fluorescent microscope (10×).

FIG. 4A depicts an exemplary fluorescent image from the expression of transgenes (fluorescent light indicated by the white arrows) in CNS tissues after tail-vein injection of recombinant AAV vectors encoding transgenes of GFP and/or human growth hormone hGH in animal models according to various disclosed embodiments of the present disclosure; and FIG. 4B depicts an exemplary fluorescent image in a control animal model after tail-vein injection of recombinant AAV vectors without any transgenes according to various disclosed embodiments of the present disclosure.

FIG. 4A shows the expression of transgenes in hippocampus of rat brain after the tail-vein injection of 100 μL of recombinant AAV vectors with the combination dosage of ⅔ of rAAV-hGH (67 μL) and ⅓ of rAAV-GFP (33 μL). For FIG. 4B, the control group was injected with 100 μL AAV vectors only. The slides (20×) were analyzed using a fluorescent microscope.

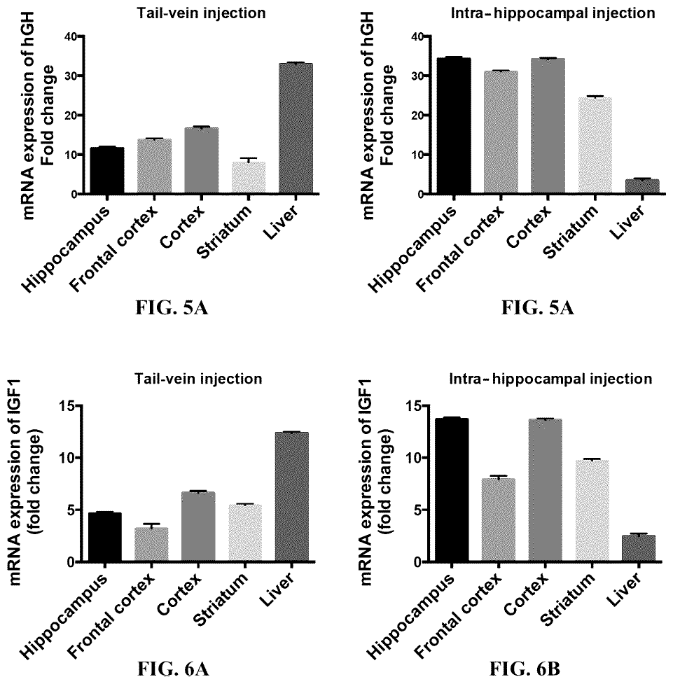

FIG. 5A depicts an exemplary quantitative-polymerase chain reaction (qPCR) detection result of mRNA expressional levels of human growth hormone hGH after the tail-vein injection according to various disclosed embodiments of the present disclosure; and FIG. 5B depicts an exemplary quantitative-PCR (qPCR) detection result of mRNA expressional levels of human growth hormone hGH after intra-hippocampal injection according to various disclosed embodiments of the present disclosure. The total RNAs were extracted from different parts of rat brain and liver, and individual cDNA library was generated by reverse transcribed (RT) reaction using a SuperScript (VLO™). Subsequently, the qPCR reaction was performed using a specific Taqman probe of human growth hormone (ABI).

In various embodiments of the present disclosure, the rat mRNA expression of hGH after intra-hippocampal and tail vein injections may be evaluated. The mRNA expression of hGH may be analyzed in four different areas of the brain (hippocampus, frontal cortex, cortex and striatum) and in the liver (FIG. 5A). Compared to the control rats, the results may demonstrate an increased mRNA expression of hGH in the liver of the tail-vein injection group. An increased hGH mRNA expression in hippocampus, frontal cortex, cortex, and striatum may also be detected in the tail-vein injection group (FIG. 5B). In the CNS injection group, compared to the control rats, an up-regulated mRNA expression of hGH may also be observed in all four brain regions. In addition, hGH mRNA expression may also be observed in the livers of these rats; and no expression of hGH may be detected in control animals.

FIG. 6A depicts an exemplary quantitative-PCR (qPCR) detection result of mRNA expressional levels of rat IGF-1 after the tail-vein injection according to various disclosed embodiments of the present disclosure; and FIG. 6B depicts an exemplary quantitative-PCR (qPCR) detection result of mRNA expressional levels of rat IGF-1 after intra-hippocampal injection according to various disclosed embodiments of the present disclosure. The cDNA library was generated from the total RNA extracted from brain tissue and liver by reverse transcribed (RT) reaction using a SuperScript (VLO™). Subsequently, the qPCR reactions were performed using a Taqman probe of rat IGF-1 (ABI). FIG. 6A may show an up-regulation after the tail-vein injection compared to the control group. FIG. 6B may demonstrate that the mRNA level of hGH in the rats given an intra-hippocampal injection was significantly up-regulated compared to the control group.

For the mRNA expression of rIGF-1 in various embodiments of the present disclosure, the mRNA expression of rIGF-1 may be analyzed in hippocampus, frontal cortex, cortex, striatum and in the liver. Compared to the controls, the results may demonstrate an increased mRNA expression of IGF-1 in hippocampus, frontal cortex, cortex striatum, and liver in the tail-vein injected rats (FIG. 6A). In the CNS injection group, compared to the controls, an up-regulated expression of IGF-1 mRNA may be found in all four brain regions analyzed (FIG. 6B). A small increase of IGF-1 mRNA expression compared to the controls may also be found in the liver.

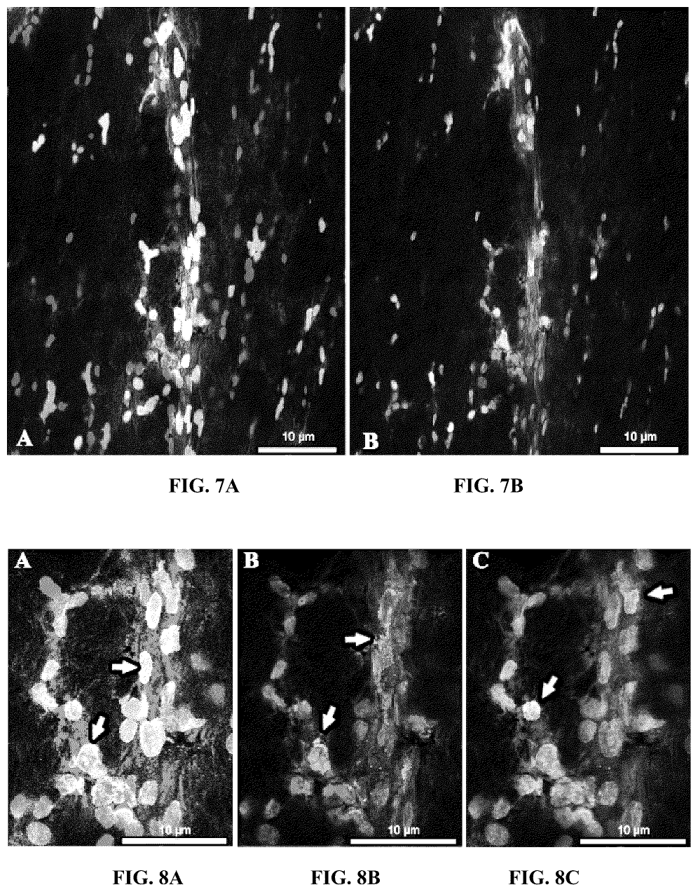

FIG. 7A depicts an exemplary fluorescent image of neurons detected by containing a specific anti-NeuN antibody with IgG conjugated to TRITC (A594/red fluorescent, 20×), where the slide was sectioned from CNS samples after the tail-vein injection, according to various disclosed embodiments of the present disclosure; and FIG. 7B depicts an exemplary fluorescent image for co-expression of hGH in the neurons by using a specific anti-human growth hormone antibody with IgG conjugated to FITC (A488/green fluorescent light, 20×), where the slide was sectioned from CNS samples after the tail-vein injection according to various disclosed embodiments of the present disclosure.

For FIG. 7A, the neurons were illustrated by a cocktail solution containing a specific anti-NeuN antibody with IgG conjugated to TRITC (A594/red fluorescent, 20×). For FIG. 7B, the expression of hGH was co-localized in the same area as the neurons, shown in FIG. 7A using a specific anti-hGH antibody with IgG conjugated to FITC (A488/green fluorescent, 20×). The slide was sectioned from samples of the tail-vein injection, stained by a cocktail solution containing double antibodies, covered by DAPI medium, and analyzed using a confocal microscope (Olympus Fluoview 1000 system).

In various embodiments of the present disclosure, co-localization of NeuN and hGH in the CNS tissues may be evaluated. Brain sections from the rats given a tail-vein injection with AAVrh10-hGH may be analyzed using immunohistochemistry. The sections may be labelled with antibodies against both neuronal marker protein of NeuN (FIG. 7A) and the hGH protein (FIG. 7B) in the hippocampus region.

The results may demonstrate co-localization of neurons labelled with NeuN and the hGH protein (FIGS. 8A-8C). FIG. 8A depicts an exemplary fluorescent image of neurons for a specific neural biomarker protein which was expressed in neurons of the CNS and detected by anti-NeuN antibody (white arrows indicate red fluorescent light), where the slide was sectioned from the CNS samples after the tail-vein injection, according to various disclosed embodiments of the present disclosure; FIG. 8B depicts an exemplary fluorescent image for the expression of hGH proteins in the cytoplasm of the neurons, detected by a specific anti-human growth hormone antibody (white arrows indicate green fluorescent staining/light), where the slide was sectioned from the CNS samples after the tail-vein injection, according to various disclosed embodiments of the present disclosure; and FIG. 8C depicts an exemplary overlapped view of FIG. 8A and FIG. 8B.

FIGS. 8A-8C illustrate co-localization of neurons with expression of hGH by double antibody-labelled immunostaining. For FIG. 8A, the neurons were illustrated by a cocktail solution containing two antibodies, one of them was a specific anti-NeuN antibody with IgG conjugated to TRITC (A594/red fluorescent, 40×). For FIG. 8B, the expression of hGH was co-localized in the same area as the neurons using a specific anti-hGH antibody with IgG conjugated to FITC (A488/green fluorescent, 40×). The data may indicate that the neural marker protein was expressed in neurons of the CNS (white arrows in FIG. 8A), and the expression of hGH proteins was only in the cytoplasm of the neurons (white arrows in FIG. 8B). The brain was sectioned from rats given the tail-vein injection, stained by a cocktail solution containing double antibodies, covered by DAPI medium, and analyzed using a confocal microscope (Olympus Fluoview 1000 system).

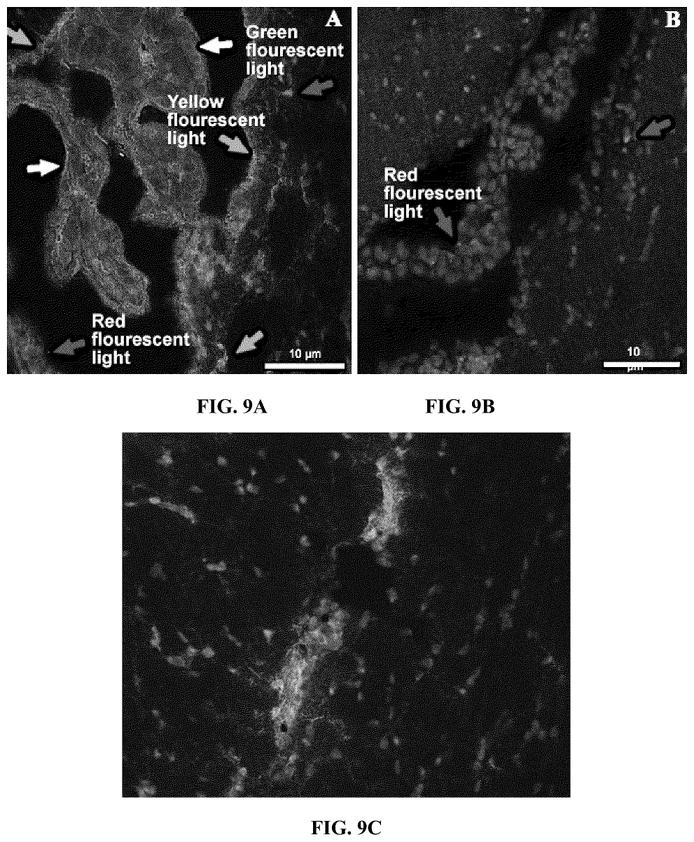

FIG. 9A depicts an exemplary fluorescent image for the expression of hGH proteins detected by a specific antibody (green fluorescent staining/light) in CNS choroid plexus, neurons identified by a specific anti-NeuN antibody (red fluorescent light), and overlapped co-localizations (yellow fluorescent light) of both hGH proteins and neurons in a same view of the tissues with the delivery of recombinant AAV vectors after tail-vein injection according to various disclosed embodiments of the present disclosure; FIG. 9B depicts an exemplary fluorescent image for a control group without hGH protein expression according to various disclosed embodiments of the present disclosure; and FIG. 9C depicts an exemplary fluorescent image for the expression of hGH proteins in another section of CNS choroid plexus tissue with the delivery of recombinant AAV vectors after tail-vein injection according to various disclosed embodiments of the present disclosure.

For FIG. 9A, the hGH proteins were detected by a cocktail solution containing two antibodies, including a specific anti-hGH antibody with IgG conjugated to FITC (A488/green fluorescent, 10×). The high level of the hGH proteins was significantly observed on the choroid plexus tissues, and overlapped with the neurons; these neurons were identified by a specific anti-NeuN antibody with IgG conjugated to TRITC (A594/red fluorescent, 10×) in both sections of A and B. FIG. 9B is served as a control (group) in same tissues of the CNS brain without expression of hGH protein. The brain was sectioned from rats given the tail-vein injection, stained by a cocktail solution containing double antibodies, covered by DAPI medium, and analyzed using a confocal microscope (Olympus Fluoview 1000 system).

It should be noted that high levels of the hGH expression (FIG. 9A, green fluorescent) may also be observed in the choroid plexus. In contrast, no detectable levels of these constituents may be found in corresponding regions of control animals (FIG. 9B).

In various embodiments of the present disclosure, the protein levels of hGH in hippocampus, frontal cortex, cortex, striatum, and in the liver may be analyzed with ELISA. In the rats given the intra-hippocampal injection with AAVRH10-hGH, expression of hGH may be observed in all four brain regions analyzed, with the highest expression in hippocampus (as shown in Table 3). Table 3 shows the Immunoassay human growth hormone levels. hGH expression may also be detected in the liver of these rats but to a lower extent. In the rats given the tail-vein injection with the AAVRH10-hGH vector, a high expression of hGH may be observed in the liver tissue. A significant expression of hGH may also be detected in the hippocampus, frontal cortex, cortex, and striatum (as shown in Table 3).

TABLE 3

| | Tail-vein | | Intracerebral | |
|---|---|---|---|---|
| | Controls (pg/ml) | hGH (pg/ml) | Controls (pg/ml) | hGH (pg/ml) |
| Hippocampus | 0.13 ± 0.03 | 165.62 ± 14.57 | 0.16 ± 0.03 | 1669.74 ± 43.11 |
| Frontal cortex | 0.15 ± 0.03 | 118.57 ± 33.37 | 0.16 ± 0.03 | 649.39 ± 33.53 |
| Cortex | 0.09 ± 0.01 | 194.52 ± 7.88 | 0.13 ± 0.02 | 861.56 ± 145.80 |
| Striatum | 0.07 ± 0.01 | 138.28 ± 26.93 | 0.08 ± 0.02 | 585.50 ± 17.66 |
| Liver | 0.25 ± 0.06 | 1067.84 ± 75.40 | 0.24 ± 0.04 | 8.12 ± 1.17 |

According to various embodiments of the present disclosure, the delivery of hGH into CNS may be able to act its biological function in both areas of CNS and peripheral system. The samples collected from brain tissues and liver may be prepared as a protein solution in a 96 well plate. Determination of hGH level may be performed according to the protocol of hGH ELISA kit (R&D System Inc) and the optical density of each well may be measured using a Fluorostar microplate reader (Molecular Devices, LLC) set at 450 nm. As shown in the Table 3 from top to the bottom are hippocampus, frontal cortex, cortex, striatum, and liver. X-axis may indicate diluted tissue samples [AG65] (1:50); and Y-axis may show the concentration of hGH level (pg/mL).

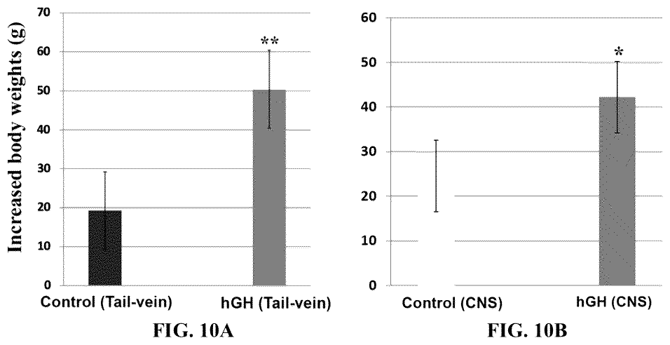

Body weight may be described in various embodiments of the present disclosure herein. To confirm the effect of the transferred hGH gene on its protein product, the body weight of the rats may be monitored throughout the experiment. FIG. 10A depicts effect of hGH treatment in tail-vein injection on body weights according to various disclosed embodiments of the present disclosure; and FIG. 10B depicts effect of hGH treatment in CNS injection on body weights according to various disclosed embodiments of the present disclosure. The weight gain may be calculated by subtracting the recorded weight at the first day from the weight at the final day of the experiment. Both the tail vein and the CNS injection groups receiving the AAVRH10-hGH vector may gain significantly more weight than the rats injected with the control vector (FIGS. 10A-10B). The change in body weight may be significantly higher (p <0.01) in the group given the tail-vein injection with AAV-hGH compared to its corresponding control group (FIG. 10A). In addition, animals treated with AAV-hGH by the intra-hippocampal injection may display a significantly increase (p<0.05) in body weight gain compared to its corresponding control group (FIG. 10B). The increase in body weight may depend on the route of vector administration as the recorded weight gain following the tail-vein injection may slightly exceed that obtained following direct intra-hippocampal injection.

The objective of the present disclosure may be to determine a desirable route for the hGH administration targeting the brain by applying gene therapy. Various embodiments of the present disclosure may indicate that the vector carrying the hGH gene that is capable to cross the BBB may be successfully constructed. Thus, the vector may be suitable for reaching hGH-responsive areas not only through direct intra-cranial injection but also through the tail-vein injection. Furthermore, it may be observed that the transferred gene may be transcribed to its message, which was subsequently translated to the protein hormone in order to elicit its biological or therapeutic effects in the CNS. It may also be observed that the hGH mediator IGF-1 may be formed as a result of hGH stimulation.

Moreover, co-localization of neurons with the expression of hGH by double antibody-labelled immunostaining may be observed. Thus, the expression of hGH may be co-localized in the same area as the neurons according to the experiment where the specific anti-NeuN antibody with IgG conjugated to TRITC (A594/red fluorescent) may be co-localized in the same area as a specific anti-hGH antibody with IgG conjugated to FITC (A488/green fluorescent). The observation may be considered to be indicative of an expression of the hGH-gene in neurons of the CNS.

The choroid plexus (CP) along with the BBB is recognized for providing a robust protective effort for the CNS, which is a physical barrier to impede entrance of toxic metabolites to the brain. It has been attributed a function related to regulation of the transport of growth factors, hormones and other medicinal agents over the BBB, as well as its preponderant role on initiating central diseases and remediation. It should be noted that high levels of hGH proteins may be also found in CP (FIGS. 9A-9C). FIG. 9A may indicate the presence of hGH binding protein on the receptor, as detected by its specific antibody (green fluorescent) in animals receiving the AAVRH10-hGH vector, whereas no binding was found in the control animal (FIG. 9B). The observation may reflect that hGH formed in the CNS may find and bind to the sites in the CP. However, hGH binding to its receptor sites in the CP may also reflect an overproduction of the hormone in peripheral tissues, such as the liver.

A further observation confirming a successful transfer of the hGH gene by the presence of the hormone protein in the rat may be indicated by the increased body mass following the injection of the AAVRH10-hGH vector as shown in FIGS. 10A-10B. Compared to the control, both CNS and tail-vein injections may cause a significant increase in body weight gain. The body weight increase following the peripheral injection through the tail vein may surpass the body weigh increase following the direct CNS administration into the hippocampus.

Regarding the detected levels of immuno-reactive hGH which are recorded by the ELISA technique, it should be noted that the hormone protein may be expressed in most brain regions examined in both intra-cerebral injection and tail vein injection. In both routes of administration of the AAVRH10-hGH vector, the hormone may be found to be detected in areas like hippocampus and liver. Compared to the controls, for the tail-vein injection, it may detect increased amounts of hGH in the brain areas hippocampus, frontal cortex, cortex and striatum.

Similar observation may be seen in the case of CNS injection, where the hormone levels may be significantly higher in all brain areas examined, particularly in the hippocampus. The detected hGH level in the liver of rats exposed to the peripheral administration of the AAVRH10-hGH vector may be significantly high compared to the expected very low levels in rats that received the intra-cerebral injection.

According to various embodiments of the present disclosure, it may demonstrate that current AAV vector may be used to transfer the hGH gene from the periphery into the brain area hippocampus. The hormone protein may be expressed and functionally active as concluded from the observation that the expression of the gene transcript of its mediator IGF-1 may be stimulated. However, it should be noted that although the hormone can reach desired areas in the brain, it may also be expressed in non-desirable areas (e.g., the liver). The expression of the GH in the liver area and in other peripheral tissue may be related to the body weight increase as observed in experimental animals. An increase may be most pronounced in animals receiving the tail-vein injection.

According to various embodiments of the present disclosure, the results may suggest that the used delivery system may not only be helpful as an investigative tool for understanding of the function of the GH-related target cells in the CNS, but also may be a valuable therapeutic implication on treatment of GH-deficient patients. However, it should be noted that the vector construct may need to be modified in a manner that the delivery of the GH gene may be limited to specific targets (e.g., relevant neurons) for desirable hormone effect.

Therefore, the construct used herein may be further developed as universal delivery system to carry other gene products to target the diseases in the CNS. The carriers may be viral gene vectors such as AAV or HIV, fusion proteins, activated domain of polypeptides or peptides. Since the GH receptors are present in the human choroid plexus, it may suggest that hGH might enter into the brain via binding to its receptors in the tissues. The binding domain of such sequence may be selected and fused with other therapeutic protein or inserted on the surface of the envelope or capsule of the vector particle. In addition, expression or production of fusion proteins with capability to cross the blood brain barrier may be designed using the vector system. Such approach may be useful for the treatment of patients with CNS disorders and other suitable diseases.

Although it is evident some viral gene vectors that delivered transgenes or therapeutic genes into the cells of the CNS have been developed, these vectors may not be able to cross the BBB. Overcoming the difficulty of delivering therapeutic agents to specific regions of the brain may present a major challenge to treatment of most brain disorders. Therefore, in the present disclosure, the vector (e.g., platform) may be developed for delivering recombinant hGH as an example of therapeutic protein or peptide to target certain tissues or cells in the CNS by crossing the BBB, which may elicit desired and sufficient therapeutic effects in the target tissues and cells of the CNS in animal models in vivo. The vector may have therapeutically implication for treatment of those patients who are suffered from neurodegenerative disorders such as those related to GH deficiency. These disorders may be from childhood onset of GHD but also from acquired GHD over the life span. For example, complications may appear during aging, but also may result from various trauma or exposure to addicted drugs. In brain injuries or trauma to the spinal cord, a decline in GH function may be repaired or restored following GH therapy, and GH therapy may also be useful for the treatment of cognitive decline resulting from long term exposure to opioids.

As disclosed above, the present disclosure provides a route for delivery of hGH to its responsive area in the brain by applying gene therapy. The AAV vector may be constructed to carry the hGH gene across the BBB. The AAV vector construct may demonstrate desirable features in experimental animals for the delivery of the gene of hGH to desired areas in the brain by not only following the intra-cranial injection but also following the tail-vein injection. It may be confirmed that the transferred gene may be transcribed to its mRNA and subsequently translated to the protein hormone, which may be capable of inducing the formation of its mediator IGF-1.

Although some embodiments of the present disclosure have been described in detail through examples, those skilled in the art should understand that the above-mentioned embodiments are only for illustration and not for limiting the scope of the present disclosure. Those skilled in the art should understand that the above-mentioned embodiments may be modified without departing from the scope and spirit of the present disclosure. The scope of the present disclosure may be defined by the appended claims.

What is claimed is:

1. An expression vector for amplified expression in mammalian cells, comprising:
one or more inserted foreign DNA sequences, including human growth hormone (hGH) and a plurality of therapeutic genes, wherein the one or more inserted foreign DNA sequences are fused with intracellular or extracellular secretion sequences;
a promoter, configured to drive expression of the one or more inserted foreign DNA sequences;
an insulator, configured for stable and safety expression of the one or more inserted foreign DNA sequences;
a marker gene, configured to encoding one or more corresponding proteins to indicate the expression of the one or more inserted foreign DNA sequences; and
a regulator element, configured to control expression of the hGH and the plurality of therapeutic genes under a controllable condition.

2. The expression vector according to claim 1, wherein the expression vector includes:
an adenoviral associate virus (AAV) vector and/or a lentiviral vector, the lentiviral vector including a human immunodeficiency virus (HIV) vector; and/or
a non-viral vector, including a lipid nanoparticle (LNP) vector.

3. The expression vector according to claim 1, wherein:
the promoter includes a cytomegalovirus (CMV) promoter or an elongation factor (EF-1) promoter or a synthetic specific promoter.

4. The expression vector according to claim 1, wherein:
the marker gene includes a green fluorescent protein (GFP) gene and/or a non-toxic mammalian biomarker.

5. The expression vector according to claim 1, further including:
a neuronal promoter, including human synapsin, configured to drive expression of transgenes in neurons in a central nervous system (CNS).

6. The expression vector according to claim 1, wherein:
the insulator includes one or more of a first chromatin insulator, a second chromatin insulator, and a third chromatin insulator, wherein:
the first chromatin insulator includes at least a part of a sequence of:
GAGCTCACGGGGACAGCCCCCCCCCAAAGCCCCCAGGGA
TGTAATTACGTCCCTCCCCCGCTAGGGGGCAGCAGCGAG
CCGCCCGGGGCTCCGCTCCGGTCCGGCGCTCCCCCCGCA
TCCCCGAGCCGGCAGCGTGCGGGGACAGCCCGGGCACGG
GGAAGGTGGCACGGGATCGCTTTCCTCTGAACGCTTCTC
GCTGCTCTTTGAGCCTGCAGACACCTGGGGGGATACGGG
GAAAAAGCTTTAGGCTGAAAGAGAGATTTAGAATGACAG
GCGCGCCTGGCCATACATCGATACGGTACCGAGTTGGCG
CGCCTGGGAGCTCACGGGGACAGCCCCCCCCCAAAGCCC
CCAGGGATGTAATTACGTCCCTCCCCCGCTAGGGGGCAG
CAGCGAGCCGCCCGGGGCTCCGCTCCGGTCCGGCGCTCC
CCCCGCATCCCCGAGCCGGCAGCGTGCGGGGACAGCCCG
GGCACGGGGAAGGTGGCACGGGATCGCTTTCCTCTGAAC
GCTTCTCGCTGCTCTTTGAGCCTGCAGACACCTGGGGGG
ATACGGGGAAAAAGCTT;
the second chromatin insulator includes at least a part of a sequence of:
CACAGGTGCTAATATTTCAATTATAGCTCTAAATCAGTGG
CCAAAAGCGTGGCCCCGACAAAAGGCTTCTATGGGCTTGA
TTGGAGTAGGCACAGCATCTGAAGTCTATCAAAGTGCTAC
AATTTTACATCGTTTAAGACCAGATGGGCAGGAAGTCACC
ATCCAACCTATGATTACTTTCATTCCCATTAATTTGTAGG
GGAGAGATTTGTTACAGCAATGGGGTACAGAAATCACCAT
TCCCTCTGCAATTTACAGTCCTGAGAGCCAAAGAATGATG
ACCAAAATGGGATATATTCCTGGGAAAGGATTAGGAAAAA
AATGAGGACGGAATAACCCAGCCGATTGAGGTCTCAGTTA
AATTTGACCGAAGAGGAATTGGATATCCTTCTTAGGTGCG
GCCACTGTTGAGCCTCCAAAGCCCATTGCATTAAAACGGA
AAACTCCAAAACCTGTTTGGGTTGATCAGTGGCCGCTTCC
TCAACAAAAACTGGAGGCATTACATTCATTGGCAAAAGAA
CAATTAGACAAAGGACATATTGAACCATCTTTTTCACCAT
GGAACGCCCCAGTTTTTGTAATTCAGAAAAAATTCGCTAG
ATGGCGCATATTGACCGACTTATGAGCGGTTAACGCAGCC
ATTCAACCGATGGGAGCTCTCCAACCAGGGTTGCCATCTC
CGGCCATGATCCCAAAAGAATGGCCGATGGTAATAATTGA
TCTAAAAGATTGTGTTTTTTACTATTCCTTTGGCTCCTCA
GGATTTTGAGAAATTTGCATTTACAATACCTGCCATGAAT
AACAAGGAACCAGCTACTATATATCGATGGAAAGTTGTAC
CCCAAGGAATGTTAAATAGTCAAACTTTTGTTGGAAAGGT
TATTCAGCTTGTCAGGGATCAGTTTCCTGATTGCTATATT
ATTCATTATGTTGATGACATTTTATGTGCTGCTGCAAGCA
GGGACAGACGGATTGAGTGTTTTGCTAGGCTACAAGAAGT
GGTGGGACTTACAGGTCTCGCTATAGCACCAGATAAAATC
CATACTACACCCTATCATTACCTGGGAATGAG;
and
the third chromatin insulator includes at least a part of a sequence of:
GAAGGTGA TGCAGTCGAA GCCATTGTGG AGGAGTCCGA
AACTTTTATTAAAGGAAAGG AGAGAAAGAC TTACCAGAGA CGCCGGGAAG
GGGGCCAGGA AGAAGATGCC TGCCACTTAC CCCAGAACCAGACGGATGGG
GGTGAGGTGG TCCAGGATGT CAACAGCAGT GTACAGATGG TGATGATGGA
ACAGCTGGAC CCCACCCTTC TTCAGATGAA GACTGAAGTA ATGGAGGGCA
CAGTGGCTCC AGAAGCAGAG GCTGCTGTGG ACGATACCCA GATTATAACT
TTACAGGTTG TAAATATGGA GGAACAGCCC ATAAACATAG GAGAACTTCA
GCTTGTTCAA GTACCTGTTC CTGTGACTGT ACCTGTTGCT ACCACTTCAG
TAGAAGAACT TCAGGGGGCT TATGAAAATG AAGTGTCTAA AGAGGGCCTT
GCGGAAAGTG AACCCATGAT ATGCCACACC CTACCTTTGC CTGAAGGGTT
TCAGGTGGTT AAAGTGGGGG CCAATGGAGA GGTGGAGACA CTAGAACAAG
GGGAACTTCC ACCCCAGGAA GATCCTAGTT GGCAAAAAGA CCCAGACTAT
CAGCCACCAG CCAAAAAAAC AAAGAAAACCAAAAAGAGCA AACTGCGTTA
TACAGAGGAG GGCAAAGATG TAGATGTGTC TGTCTACGAT TTTGAGGAAG
AACAGCAGGA GGGTCTGCT TCAGAGGTTA ATGCAGAGAA AGTGGTTGGT
AATATGAAGC CTCCAAAGCC AACAAAAATT AAAAAGAAAG GTGTAAAGAA
GACATTCCAG TGTGAGCTTT GCAGTTACAC GTGTCCACGG CGTTCAAATT
TGGATCGTCA CATGAAAAGC CACACTGATG AGAGACCACA CAAGTGCCAT
CTCTGTGGCA GGGCATTCAG AACAGTCACC CTCCTGAGGA ATCACCTTAA
CACACACACA GGTACTCGTC CTCACAAGTG CCCAGACTGC GACATGGCCT
TTGTGACCAG TGGAGAATTG GTTCGGCATC GTCGTTACAA ACACACCCAC
GAGAAGCCAT TCAAGTGTTC CATGTGCGAT TACGCCAGTG TAGAAGTCAG
CAAATTAAAA CGTCACATTC GCTCTCATAC TGGAGAGCGT CCGTTTCAGT
GCAGTTTGTG CAGTTATGCC AGCAGGGACA CATACAAGCT
GAAAAGGCACATGAGAACCC ATTCAGGGGA AAAGCCTTAT GAATGTTATA
TTTGTCATGC TCGGTTTACC CAAAGTGGTA CCATGAAGAT GCACATTTTA
CAGAAGCACA CAGAAAATGT GGCCAAATTT CACTGTCCCC ACTGTGACAC
AGTCATAGCC CGAAAAAGTG ATTTGGGTGT CCACTTGCGA AAGCAGCATT
CCTATATTGA GCAAGGCAAG AAATGCCGTT ACTGTGATGC TGTGTTTCAT
GAGCGCTATG CCCTCATCCA GCATCAGAAG TCACACAAGA ATGAGAAGCG
CTTTAAGTGT GACCAGTGTG ATTACGCTTG TAGACAGGAG AGGCACATGA
TCATGCACAA GCGCACCCAC ACCGGGGAGA AGCCTTACGC CTGCAGCCAC
TGCGATAAGA CCTTCCGCCA GAAGCAGCTT CTCGACATGC ACTTCAAGCG
CTATCACGAC CCCAACTTCG TCCCTGCGGC TTTTGTCTGT TCTAAGTGTG
GGAAAACATT TACACGTCGG AATACCATGG CAAGACATGC TGATAATTGT
GCTGGCCCAG ATGGCGTAGA GGGGGAAAAT GGAGGAGAAA CGAAGAAGAG
TAAACGTGGA AGAAAAAGAA AGATGCGCTC TAAGAAAGAA GATTCCTCTG
ACAGTGAAAA TGCTGAACCAGATCTGGACG ACAATGAGGA TGAGGAGGAG
CCTGCCGTAG AAATTGAACC TGAGCCAGAGCCTCAGCCTG TGACCCCAGC
CCCACCACCC GCCAAGAAGC GGAGAGGACG ACCCCCTGGCAGAACCAACC
AGCCCAAACA GAACCAGCCA ACAGCTATCA TTCAGGTTGA AGACCAGAAT
ACAGGTGCAA TTGAGAACAT TATAGTTGAA GTAAAAAAAG AGCCAGATGC
TGAGCCCGCA GAGGGAGAGG AAGAGGAGGC CCAGCCAGCT GCCACAGATG
CCCCCAACGG AGACCTCACG CCCGAGATGA TCCTCAGCAT GATGGACCGG

7. The expression vector according to claim 2, wherein:
a surface capsule of the expression vector is engineered by inserting a nucleotide sequence to bind and/or recognize one or more receptors on Choroid plexus and blood brain barrier (BBB) tissues.

8. The expression vector according to claim 2, wherein:
a surface capsule of the expression vector is engineered by inserting a single-chain fragment variable (scFv) antibody sequence to bind and/or recognize one or more receptors on Choroid plexus and blood brain barrier (BBB) tissues.

9. The expression vector according to claim 2, wherein:
a surface capsule of the expression vector is engineered by inserting a ligand sequence derived from the hGH to bind and/or recognize one or more hGH receptors on Choroid plexus and blood brain barrier (BBB) tissues.

10. The expression vector according to claim 1, wherein:
a biological tracer is used as an investigative and/or diagnostic tool to mark cells and/or to generate a specific animal model in vivo, and/or the biological tracer is applied for diagnostic instruments.

11. The expression vector according to claim 1, wherein:
the one or more inserted foreign DNA sequences are genes encoding proteins, peptides and transcription factors including IGF-1 (insulin growth factor-1), insulin, rat growth hormone, glucocerebrosidease, brain-derived neurotrophic factor (BDNF), β-endorphin, HSV-1 (herpes simplex virus), anti-glioma, ApoE4, presenilin 1 and/or 2, myelin oligodendrocyte glycoprotein (MOG), crry, gata4, glutamate decarboxylase 65, interleukin-10, chondroitinase ABC, P/NK-1R, human interleukin-12 (hIL-12), stem cell transcription factors for iPSC including October 3/4, Sox2, Klf1-5, Nanog, LIN28 and GIis1, direct dopaminergic neurons reprogramming including ASCl1, LMX1A, MYT1L and BRN2, direct glutamatergic, GABAergic neuronal reprogramming including ASCl1, MYT1L, BRN2. and NeuroD1, direct retinal pigment epithelium reprogramming including PAX6, RAX, CRX, MITF-A, OTX2, NRL and KLF4/2, and direct spinal motor neurons reprogramming including NGN2, SOX11, ISL1 and LHX3, and further including anti-Tau, anti-β-amyloid antibodies, and nanobodies (VHH).

12. The expression vector according to claim 1, wherein:
the one or more inserted foreign DNA sequences include CRISPR/Cas9, antisense, sense, or mRNA used for treating a central nervous system (CNS) disease.

13. The expression vector according to claim 1, wherein:
the one or more inserted foreign DNA sequences include antisense or sense used for treating a central nervous system (CNS) disease, the CNS disease including brain viral infection.

14. The expression vector according to claim 1, wherein:
the one or more inserted foreign DNA sequences include shRNA, RNAi, or mRNA used for treating a central nervous system (CNS) disease, the CNS disease including brain viral infection.

15. The expression vector according to claim 1, wherein:
the expression vector is used to treat a central nervous system (CNS) disease including brain viral infection by delivering the hGH and the plurality of therapeutic genes via crossing the BBB.

16. The expression vector according to claim 15, wherein:
delivering the hGH and the plurality of therapeutic genes into the CNS via crossing the BBB is administrated with intravenous injection formulation, nasal spray formulation, inhalation formulation, liquid and/or tablet formulations, eye drop administration formulation, or a combination thereof.

17. The expression vector according to claim 1, wherein the regulator element includes:
(1) a tetracycline response element (TRE) including sequences of “TCCCTATCAGTGATAGAGA” to be included into the vector; and/or
(2) a post-transcriptional regulatory element (PRE)+derived from woodchuck hepatitis virus (WHV) into 3′ untranslated region of the AAV vector or lentiviral vector to enhance both titer and transgene expression.

18. The expression vector according to claim 1, wherein:
the intracellular or extracellular secretion sequences include fusion sequences, the fusion sequences including ROIKIWFONRR, YGRKKRRORRR, and/or LLNFDLLKILLAGDWESNPCP.

19. A method for generating an expression vector for amplified expression in mammalian cells, comprising:
fusing one or more inserted foreign DNA sequences with intracellular or extracellular secretion sequences, the one or more inserted foreign DNA sequences including human growth hormone (hGH) and a plurality of therapeutic genes;
configuring a promoter to drive expression of the one or more inserted foreign DNA sequences;
configuring an insulator for stable and safety expression of the one or more inserted foreign DNA sequences;
configuring a marker gene to encoding one or more corresponding proteins to indicate the expression of the one or more inserted foreign DNA sequences; and
configuring a regulator element control expression of the hGH and the plurality of therapeutic genes under a controllable condition.
